(19) United States
(12) Patent Application Publication
(10) Pub. No.: US 2021/0391054 A1
(43) Pub. Date: Dec. 16, 2021

(54) SYSTEM AND METHOD OF MANAGING GROCERY CART BASED ON HEALTH INFORMATION

(71) Applicant: GIAN CORPORATION, Pittsford, NY (US)

(72) Inventors: Jean-Michel Guillemin LABORNE, Pittsford, NY (US); Ameet BHATTACHARYA, Fairport, NY (US)

(21) Appl. No.: 17/395,511

(22) Filed: Aug. 6, 2021

Related U.S. Application Data
(63) Continuation of application No. PCT/US2020/017187, filed on Feb. 7, 2020.
(60) Provisional application No. 62/802,481, filed on Feb. 7, 2019.

(51) Int. Cl. G16H 20/60; G16H 10/60; G16H 50/70; G16H 50/30; G16H 50/20; G16H 15/00

(52) U.S. Cl. CPC G16H 20/60; G16H 10/60; G16H 50/70; G06Q 30/0633; G16H 50/20; G16H 15/00; G16H 50/30

(57) ABSTRACT

A health cart management system may be configured to generate a health cart score based on items in a grocery cart. The system may receive item information including a quantity and a sharing parameter for an item in the cart. The system may receive profile information including medical conditions of a consumer of the items. The system may receive dietary reference intake information of nutrients, compute a daily consumption value for each item in the cart, and generate a nutrient score for each nutrient. The system may generate the nutrient score by adjusting the recommended intake value of the nutrient based on the consumer's medical conditions, and computing a daily nutrient consumption value for the nutrient based on the daily consumption quantity values for all items in the cart. The system may generate the health cart score by computing a weighted mean of the nutrient scores for all nutrients.

### CROSS-REFERENCE TO RELATED APPLICATIONS

This application is a Continuation Application of International Application No. PCT/US2020/017187, filed Feb. 7, 2020, which claims the benefit of U.S. Provisional Application No. 62/802,481, filed Feb. 7, 2019, the entire disclosure of both of these applications is hereby incorporated herein by reference.

### TECHNICAL FIELD

The present invention is generally related to the field of managing a grocery cart for a user based on health data of the user and nutritional information of the items in the grocery cart.

### BACKGROUND

Changes in diet and poor nutrition choices over the past thirty years have increased the prevalence of lifestyle-related chronic diseases such as obesity, heart disease, hypertension, and diabetes. For example, more than half the U.S adult population, 100 million adults, has prediabetes or diabetes. 75% of healthcare costs in the US are due to chronic health conditions. Health care costs for people with a chronic condition average $6,032 annually—5× when compared to a healthy individual. Fortunately, advances in nutrition science, policy and technology are providing a road map on how to address this national nutrition crisis. The ‘Food Is Medicine’ movement is increasing awareness in our society of the role that food plays in health & wellness (including disease prevention). However, one prevailing issue that only continues to get worse is confusion around what to eat given all the choices available to us—people are looking for help to make healthier choices for themselves.

This need has created an enormous industry around personalized nutrition—a number of companies have emerged to help personalize a consumer's health & nutrition. Capabilities range from creation of personalized meal plans based on a person's microbiome data & dietary needs to helping make more informed healthier grocery purchase decisions.

For example, Kroger's OptUp™ platform is a smart phone application that puts nutritional information at a shopper's fingertips and helps them buy healthier products. Key features of OptUp™ include scoring groceries bought by the shopper after the fact (OptUp™ score) using nationally recognized dietary guidelines, providing personalized product recommendations, providing a household OptUp™ score, scanning and researching items to find nutrition facts and product alternatives, and adding items to a shopper's cart for curbside delivery or pickup. The OptUp™ platform keeps track of a shopper's purchases via the Kroger's shopper's card and calculates the OptUp™ score by averaging a product nutrition score for items purchased over a period of eight weeks. Scores range from 0 to 1000—the higher the score, the more nutritious the purchases. Kroger's dieticians set an OptUp™ target score to help provide guidance on healthy goals that a person would like to either maintain or achieve. For any previously purchased item, consumers have the ability to view nutrition ratings from 1 to 100, get nutrition facts, and see healthier alternatives, which can be sent to an electronic cart for curbside pickup or delivery. OptUp™ breaks nutrition ratings into different ranges. A product nutrition score of 71+ defines “better-for-you foods” that are lower in saturated fat, sodium, sugar, and calories, and may be higher in fiber, protein or ingredients like fruits, nuts and vegetables. A product nutrition score of 36 to 70 defines foods that are in the middle and somewhat higher in saturated fat, sodium, sugar, or calories, or may have lower fiber, protein, and fruit or veggie content compared to green foods in the same category. A product nutrition score of 1 to 35 identifies foods to be consumed occasionally with higher saturated fat, sodium, sugar, or calories, and lower fiber, protein, and fruit or vegetable ingredients. OptUp™, however, cannot provide a score for a shopping cart in real time or provide a shopper with healthier alternatives for items in their shopping cart.

Another known prior art system is Yuka™, a smart phone application developed in France in 2017, to help consumers choose healthy products at the point of purchase. Yuka™ works by using the smartphone camera to scan barcodes of food and personal care products. Each product is subsequently rated and detailed information, including a list of its components, is provided to help consumers understand their health. Healthier alternatives are recommended for any product deemed to have a negative impact on the consumer's health. Product information is populated using the Open Food Facts database under open license. Food product analysis is based on three criteria: nutritional quality, presence of additives and its organic aspect. Nutritional quality represents 60% of the score and is based on the European NutriScore calculation methodology. Presence of additives represents 30% of the score. Organic aspect represents 10% of the score, and is based on whether or not the product has the European organic label

Healthier alternatives are recommended using an algorithm that incorporates the following factors: product category—a product most similar to the original item is recommended; product rating—only highly rated products are recommended; product availability—only recommends products that can easily be found in stores.

However, none of the applications mentioned above and available in the market to date assess a collection of food products in a shopping cart in real time before they are purchased as part of the ecommerce workflow and provide healthier recommendations to purchase instead that take into account the consumers & their family's clinical condition and lifestyle bringing in data from clinical systems, wearables and FDA approved connected devices. Further, conventional systems rely on the nutrition facts label to score a product or a collection of products. None of the applications mentioned above utilize personalized DRI recommendations to generate personalized scores based on the user. Moreover, none of the prior art systems assess an individual's daily consumption of each food item as part of scoring the shopping cart.

### SUMMARY

At least the above-discussed need is addressed and technical solutions are achieved in the art by various embodiments of the present invention. Some embodiments of the present invention pertain to a health cart, a personalized grocery shopping cart, which is a nutritional instrument providing easy-to-understand guidance for any user, no matter their education level or nutrition knowledge. In one embodiment, the health cart provides a single score number summarizing the food positives and negatives that people are putting into their grocery shopping cart, averaged over time to show their daily intake. In some embodiments, data such as Basal Metabolic Rate (BMR), medical information, and fitness activity are incorporated into the health cart, so that the health cart is personalized, to help individuals make positive adjustments in their eating habit. To further personalize the nutrition recommendations, the health cart may receive medical record data including diagnosis, allergies, and medications, as well activity data from wearables such as FitBit, Google Fit, or FDA approved connected devices such as blood pressure monitors and glucometers.

The Health Cart Management System (HCMS) is based on a set of considered nutrients that compose most food. Both, predominant macronutrients such as fat, carbohydrate and protein, and micronutrients such as minerals and vitamins, are evaluated for each food item in the health cart. The respective quantity of each nutrient sums up to yield a total weight for every nutrient contained in a whole meal or grocery shopping cart.

The HCMS system is particularly suited to, but not limited to, users suffering from chronic diseases such as diabetes, obesity, hypertension, among other health issues. Based on chronic disease, each nutrient is set in the HCMS with a specific ponderation coefficient (weight) that determines its importance within the overall scoring computation. The value of these coefficients depends upon an individual's diagnoses, giving input to the HCMS system to compute the health cart score. For example, carbohydrate and sugar nutrients may be more critical for a diabetic's health than for a healthy person. Therefore, in some embodiments, these two nutrients may be configured with higher coefficients for diabetic users.

In some embodiments, a method executed by a programmed data processing device system of a cart management system may include receiving item information of one or more items in the cart, the item information including a quantity of each item of the one or more items in the cart and a sharing parameter for at least one item of one or more items in the cart; receiving profile information of a consumer of the one or more items in the cart, the profile information including diagnostic information of at least one medical condition of the consumer; receiving dietary reference intake information of one or more nutrients, the dietary reference intake information including a recommended intake value for each nutrient of one or more nutrients; computing a daily consumption value for each item of the one or more items in the cart; generating, for each nutrient of the one or more nutrients, a nutrient score by adjusting the recommended intake value of the nutrient based at least on the diagnostic information of the at least one medical condition of the consumer, computing a daily nutrient consumption value for the nutrient based on all items in the cart and the respective daily consumption quantity values for all items in the cart, and generating the nutrient score based at least on the daily nutrient consumption value for the nutrient and the adjusted recommended intake value of the nutrient; and generating the health cart score by computing a weighted mean of the nutrient scores for each nutrient of the one or more nutrients.

In some embodiments, the method may further comprise providing an indication of the generated health cart score to the consumer. In some embodiments, the method may further comprise outputting a health cart document including at least the generated health cart score, the nutrient scores for each nutrient of the one or more nutrients, and the adjusted recommended intake value for each nutrient of the one or more nutrients.

In some embodiments, the sharing parameter includes at least one of a family sharing factor or a sharing option. In some embodiments, the step of receiving dietary reference intake information of one or more nutrients may include adjusting the dietary reference intake information based at least on one factor of one or more factors included in the profile information of the consumer of the one or more items in the cart, the one or more factors including an age of the consumer, a weight or the consumer, a height of the consumer, an activity level of the consumer, or a basal metabolic rate (BMR) of the consumer.

In some embodiments, the step of adjusting the recommended intake value of the nutrient may include computing an eta-coefficient to increase or decrease the recommended intake value of the nutrient by a predetermined factor based on the diagnostic information of the at least one medical condition of the consumer.

In some embodiments, the step of computing the daily consumption value for each item of the one or more items in the cart may include, for each item, in a case where the item information does not include a sharing parameter for an item of the one or more items in the cart, setting a daily consumption quantity value of the item to the quantity of the item, and in a case where the item information includes a sharing parameter for an item of the one or more items in the cart, computing the daily consumption quantity value for the item based at least on the quantity of the item and the sharing parameter for the item.

In some embodiments, the method may further comprise identifying at least one deficient nutrient based on the nutrient scores of the one or more nutrients, identifying at least one item of the one or more items in the cart contributing to the nutrient score of the deficient nutrient, replacing the identified item in the cart with another item to improve the nutrient score of the deficient nutrient, and recalculating the health cart score.

In some embodiments, the method may further comprise computing an item retention for each item of the one or more items in the cart, each item retention defining a consumption period for a respective item of the one or more items in the cart, and adjusting the daily consumption quantity value of each item of the one or more items in the cart based at least on the respective item retention. In some embodiments, the step of computing the item retention for each item of the one or more items in the cart may include, in a case where a purchase history of the item is available, computing the item retention based at least on the purchase history of the item, and, in a case where the purchase history of the item is unavailable, computing the item retention based at least on a serving size of the item.

In some embodiments, a health cart management system may be summarized as including an input-output device system communicatively connected to a display device system, a memory device system storing a program, and a data processing device system communicatively connected to the input-output device system and the memory device system. In some embodiments, the data processing device system may be configured at least by the program at least to receive item information of one or more items in the cart, the item information including a quantity of each item of the one or more items in the cart and a sharing parameter for at least one item of one or more items in the cart; receive profile information of a consumer of the one or more items in the cart, the profile information including diagnostic information of at least one medical condition of the consumer; receive dietary reference intake information of one or more nutrients, the dietary reference intake information including a recommended intake value for each nutrient of one or more nutrients; compute a daily consumption value for each item of the one or more items in the cart; generate, for each nutrient of the one or more nutrients, a nutrient score by adjusting the recommended intake value of the nutrient based at least on the diagnostic information of the at least one medical condition of the consumer, computing a daily nutrient consumption value for the nutrient based on all items in the cart and the respective daily consumption quantity values for all items in the cart, and generating the nutrient score based at least on the daily nutrient consumption value for the nutrient and the adjusted recommended intake value of the nutrient; and generate the health cart score by computing a weighted mean of the nutrient scores for each nutrient of the one or more nutrients.

In some embodiments, the user's daily consumption of food items is assessed using either past purchases data or algorithms assessing food retention. In some embodiments, the daily food consumption can be adjusted, either to take into account the purchased food shared by several members of a household, or, conversely, to account for consumed groceries or meals that are purchased outside the HCMS and therefore could elude the HCMS. In some embodiments, each nutrient may be given an individual score in the health cart. In some embodiments, the overall score may be a weighted mean of the nutrient scores. Separate nutrient scores may help to identify food concerns with regard to particular nutrients. In some embodiments, the HCMS system may perform a “healthy swap” by substituting food items with healthier options. In some embodiments, food items contributing to nutrients in excess are identified. In some embodiments, foods in specific categories are suggested to intentionally increase the intake of deficient nutrients. In some embodiments, an optimization algorithm permits the HCMS system to elevate poor nutrient scores, thereby improving the overall health cart score. In some embodiments, the healthiness of an initial grocery shopping cart or a meal can be automatically optimized by the HCMS without any user's intervention.

In some embodiments, nutrient scores are determined according to a simple principle: the farther off a value is from recommended targets, the lower the score. The recommended daily targets for most nutrients are generally known. For example, tables such as the Dietary Reference Intake (DRI) tables released by the US Institute of Medicine and other international nutrition authorities such as Worldwide Health Organization (WHO) may be utilized by the HCMS. In some embodiments, the DRI values may be supplemented with lower and upper limits on the values. In some embodiments, DRI tables are set as a reference intake for healthy users. In some embodiments, a η coefficient, may be used to adjust the reference intake value of one or more nutrients for users suffering from one or more chronic diseases. For example, it is known that sodium is not healthy for hypertensive people. In this example, the η coefficient associated with sodium may be adjusted to 0.65, which means an adequate intake for hypertensive people is 35% less as compared to the adequate intake for healthy people. If appropriate, the upper limit can be lowered as well.

In some embodiments, comorbidity, where a person may suffer from more than one chronic disease, may be handled by computing specific nutrient ponderation and η coefficients that are reformulated based on nutrient ponderation and η coefficients assigned specifically to each disease individually.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 shows a computing device system, according to some embodiments of the invention.

FIG. 2 shows another computing device system, according to some embodiments of the invention.

FIG. 3 shows a diagram of the landscape in which the HCMS system operates, according to some embodiments of the present invention;

FIG. 4 shows some exemplar data stores associated with the HCMS system, according to some embodiments of the present invention;

FIG. 4A shows an exemplar data model for storing dietary reference intake data, according to some embodiments of the present invention;

FIG. 4B shows an exemplar data model for storing nutrient coefficients, according to some embodiments of the present invention;

FIG. 4C shows an exemplar data model for storing food and nutrient composition data, according to some embodiments of the present invention;

FIG. 4D shows an exemplar data model for storing multiple food taxonomies, according to some embodiments of the present invention;

FIG. 5 shows an exemplar source dietary reference intake table issued by the US Institute of Medicine, according to some embodiments of the present invention;

FIG. 6 shows an exemplar data structure representing a Health Cart, according to some embodiments of the present invention;

FIG. 7 is a flowchart describing a method of generating a Health Cart document, according to some embodiments of the present invention;

FIG. 7A shows an exemplar data structure of a shopping cart, according to some embodiments of the present invention;

FIG. 7B shows an exemplar data structure of a shopper's profile, according to some embodiments of the present invention;

FIGS. 8A-8C show exemplar functors for computing nutrient scores, according to some embodiments of the present invention;

FIG. 9A shows exemplar score bar visualizations of an overall Health Cart score, according to some embodiments of the present invention;

FIG. 9B shows exemplar score bar visualizations of a neutral nutrient bar score, according to some embodiments of the present invention;

FIG. 9C shows exemplar score bar visualizations of a low-is-better nutrient bar score, according to some embodiments of the present invention;

FIG. 9D shows exemplar score bar visualizations of a high-is-better nutrient bar score, according to some embodiments of the present invention;

FIG. 9E shows an exemplar representation of the seed geometry to shape any score bars, according to some embodiments of the present invention;

FIG. 10 shows an exemplar function for determining whether to nudge a nutrient, according to some embodiments of the present invention;

FIG. 11 shows an exemplar user interface permitting a shopper to share a shopping cart item across other household members, according to some embodiments of the present invention;

FIG. 12A shows an exemplar user interface to alert a shopper of a nutritional issue with a shopping cart item and trigger the Healthy Swap feature, according to some embodiments of the present invention;

FIG. 12B shows an exemplar user interface to display healthier alternative foods as an outcome of the Healthy Swap process, according to some embodiments of the present invention;

FIG. 13A shows an exemplar user interface of a Health Cart with two examples of deficient nutrients, according to some embodiments of the present invention;

FIG. 13B shows an exemplar user interface to suggest food categories to improve a deficient nutrient, according to some embodiments of the present invention;

FIG. 14 shows a visual representation of a shopper's food categorization, according to some embodiments of the present invention;

FIG. 15 is a flowchart showing a method of computing food item retention, according to some embodiments of the present invention;

FIG. 15A is a flowchart showing a method of computing the energy of mutually exclusive items (MEIs) contained in a shopping cart, according to some embodiments of the present invention;

FIG. 15B is a flowchart showing a method of computing the residual basal metabolic rate (rBMR), energy left to be consumed by the MEIs, according to some embodiments of the present invention;

FIG. 15C is a flowchart showing a method of determining the retention of a shopping cart item whose purchase history is available, according to some embodiments of the present invention;

FIG. 15D is a flowchart showing a method of determining the retention of a shopping cart item without purchase history, according to some embodiments of the present invention;

FIG. 16 shows an exemplar user interface to add family members to a user profile, according to some embodiments of the present invention;

FIG. 17 is a flowchart showing a method for substituting multiple items in a Health Cart, according to some embodiments of the present invention;

FIG. 18 is a flowchart showing a method for optimizing a Health Cart, according to some embodiments of the present invention;

FIG. 19 is a flowchart showing a method for seeking healthier alternative food items in a Health Cart, according to some embodiments of the present invention;

FIG. 20A shows an exemplar food taxonomy, according to some embodiments of the present invention; and

FIG. 20B shows another exemplar food taxonomy, according to some embodiments of the present invention.

### DETAILED DESCRIPTION

In some embodiments, the HCMS (Health Cart Management System) solution provides easy-to-understand guidance for managing a user's shopping cart, irrespective of their educational level or nutrition knowledge. It should be noted that the invention is not limited to these or any other examples provided herein, which are referred to for purposes of illustration only.

In this regard, in the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, some embodiments of the present invention may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

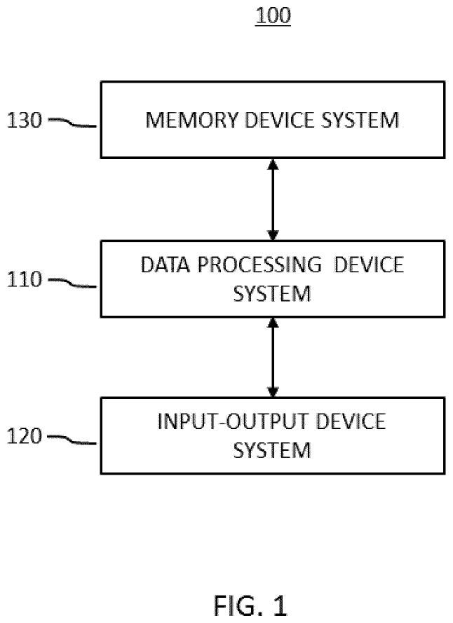

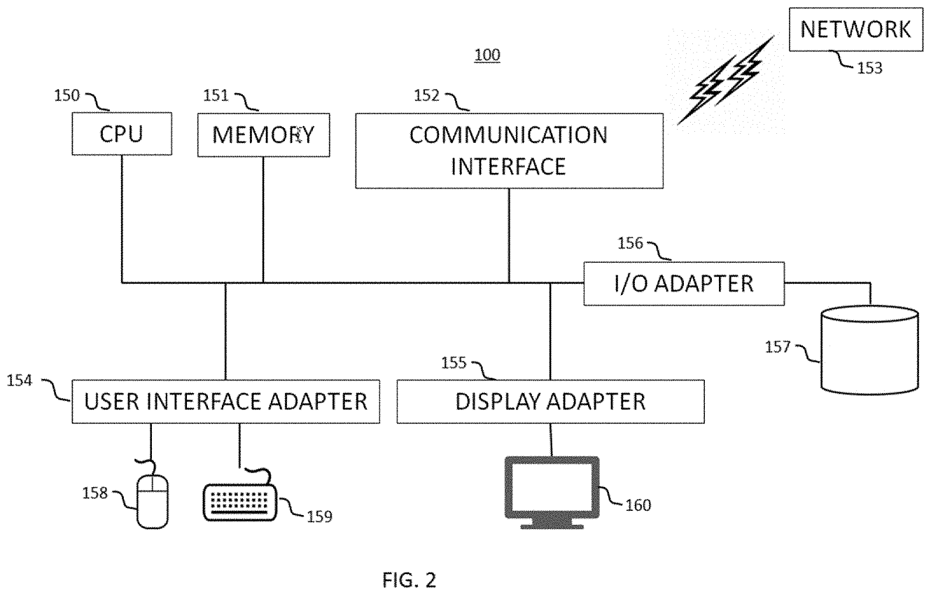

The term “program” in this disclosure should be interpreted to include one or more programs including as a set of instructions or modules that may be executed by one or more components in a system, such as a controller system or data processing device system, in order to cause the system to perform one or more operations. The set of instructions or modules may be stored by any kind of memory device, such as those described subsequently with respect to the memory device system 130, 151, or both, shown in FIGS. 1 and 2, respectively. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

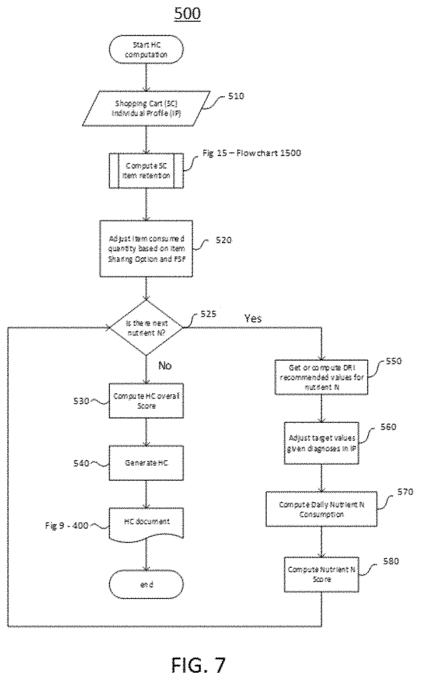

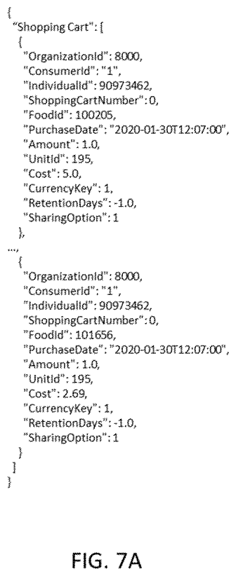

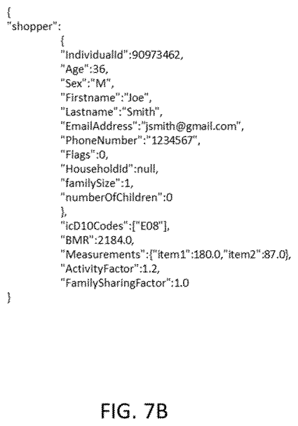

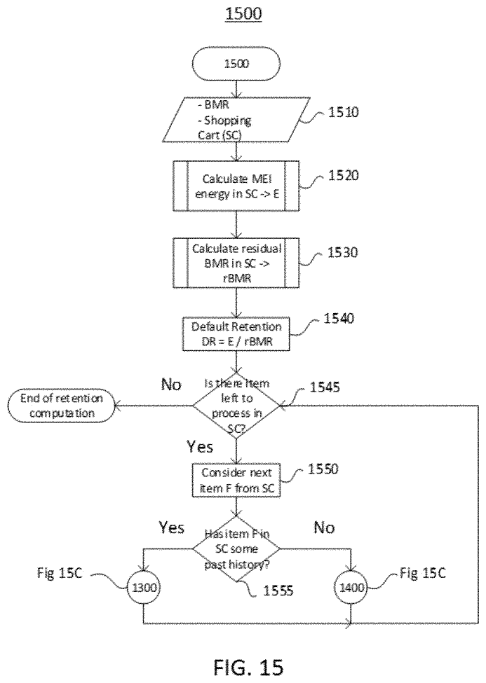

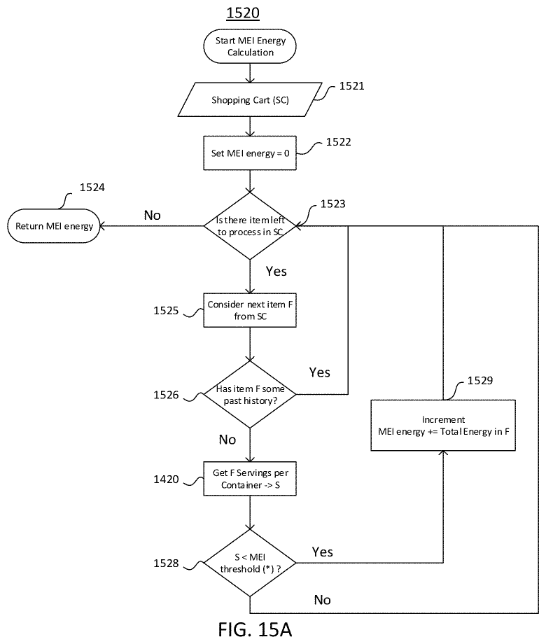

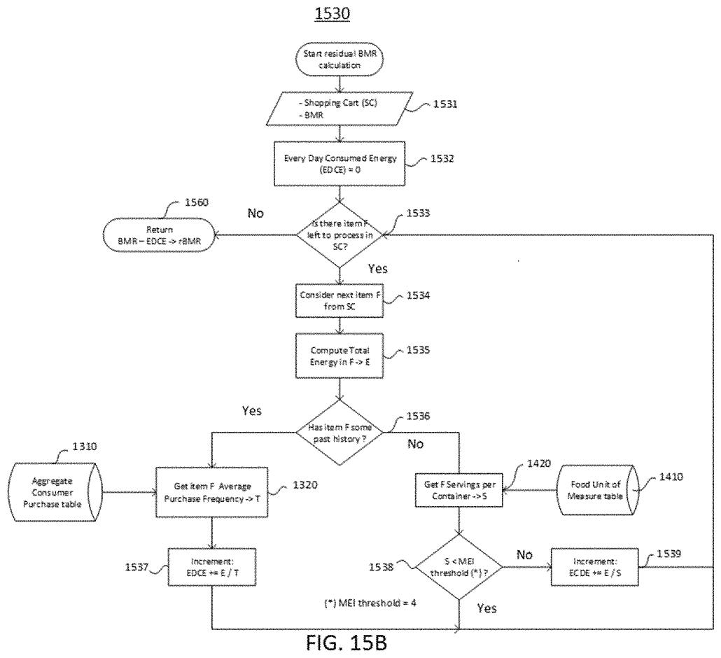

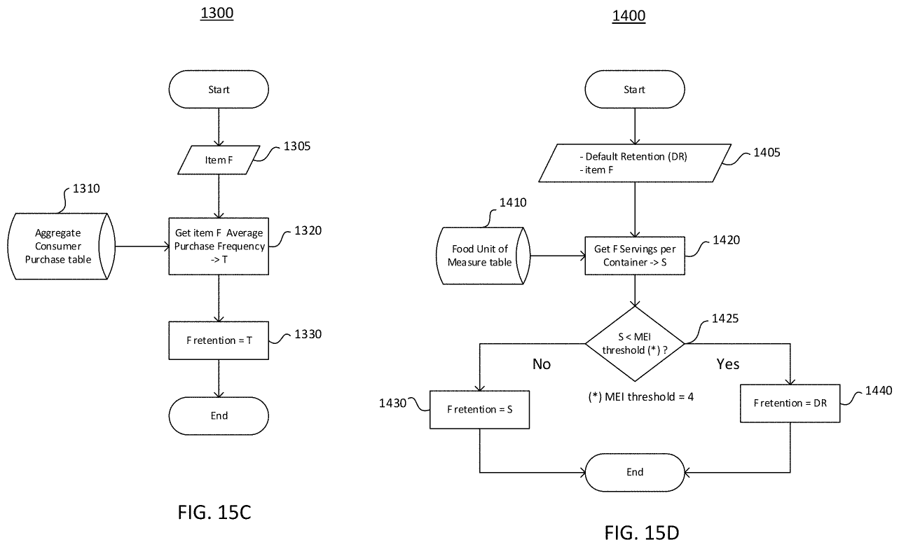

Further still, example methods are described herein with respect to FIGS. 7, 15, 15A-D, and 17-19. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangement shown in method FIGS. 7, 15, 15A-D, and 17-19 herein is not limited to an actual structure of any program or set of instructions or required ordering of method tasks, and such method FIGS. 7, 15, 15A-D, and 17-19, according to some embodiments, merely illustrates the tasks that instructions are configured to perform, for example upon execution by a data processing device system in conjunction with interactions with one or more other devices or device systems.

FIG. 1 schematically illustrates a system 100 according to some embodiments. In some embodiments, the system 100 may be a computing device 100 (as shown in FIG. 2). In some embodiments, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various embodiments. Each of the phrases “data processing device”, “data processor”, “processor”, and “computer” is intended to include any data processing device, such as a central processing unit (“CPU”), a desktop computer, a laptop computer, a mainframe computer, a tablet computer, a personal digital assistant, a cellular phone, and any other device configured to process data, manage data, or handle data, whether implemented with electrical, magnetic, optical, biological components, or other.

The memory device system 130 includes one or more processor-accessible memory devices configured to store information, including the information needed to execute the control programs associated with some of the various embodiments. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some embodiments, each of the phrases “processor-accessible memory” and “processor-accessible memory device” is intended to include a non-transitory computer-readable storage medium. In some embodiments, the memory device system 130 can be considered a non-transitory computer-readable storage medium system.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the embodiments.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the embodiments. In this regard, the input-output device system may include various other devices or systems described in various embodiments.

FIG. 2 shows an example of a computing device system 100, according to some embodiments. The computing device system 100 may include a processor 150, corresponding to the data processing device system 110 of FIG. 1, in some embodiments. The memory 151, input/output (I/O) adapter 156, and non-transitory storage medium 157 may correspond to the memory device system 130 of FIG. 1, according to some embodiments. The user interface adapter 154, mouse 158, keyboard 159, display adapter 155, and display 160 may correspond to the input-output device system 120 of FIG. 1, according to some embodiments. The computing device 100 may also include a communication interface 152 that connects to a network 153 for communicating with other computing devices 100.

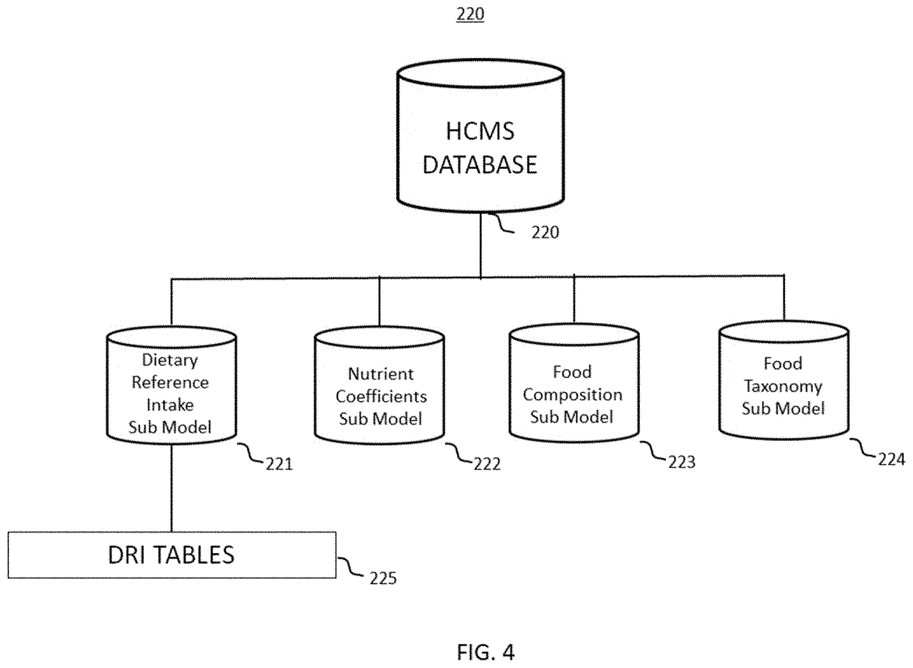

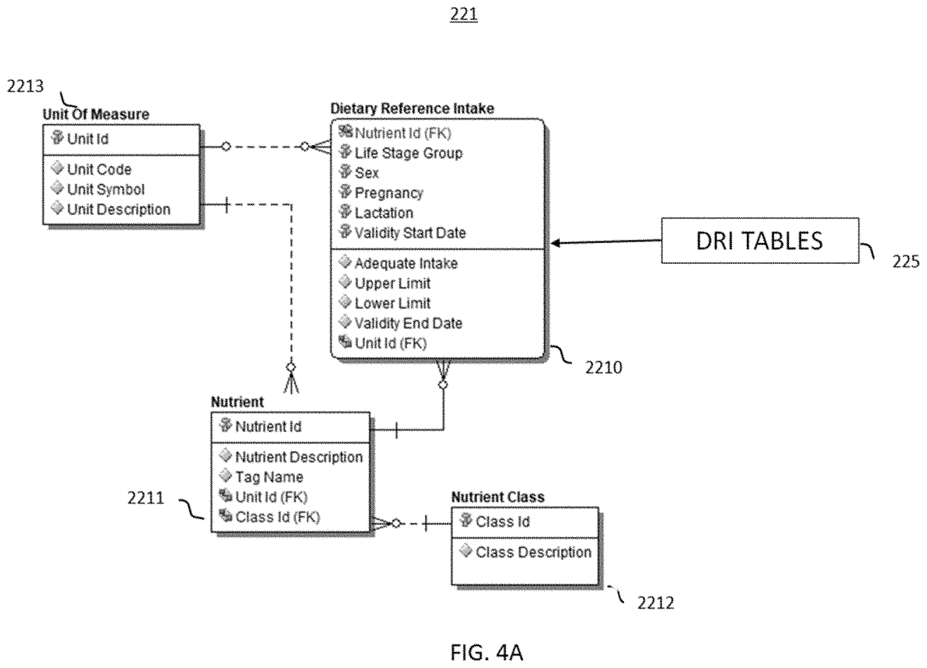

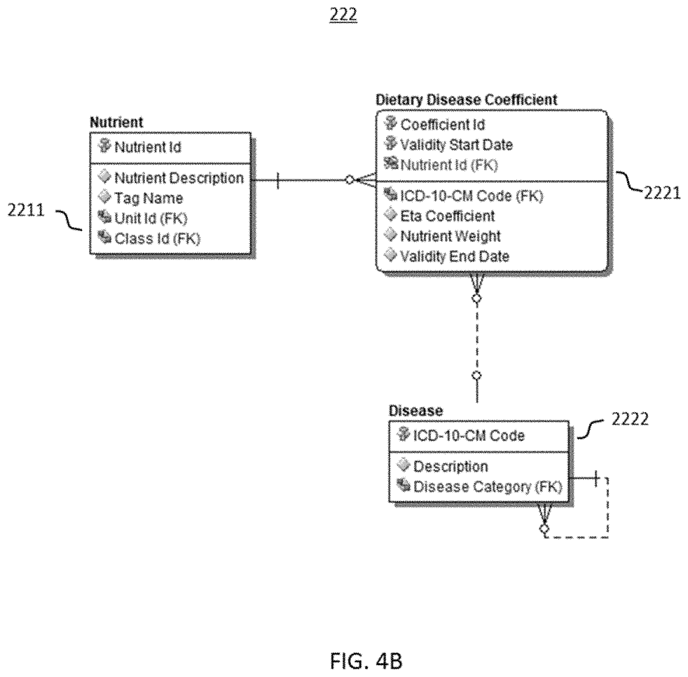

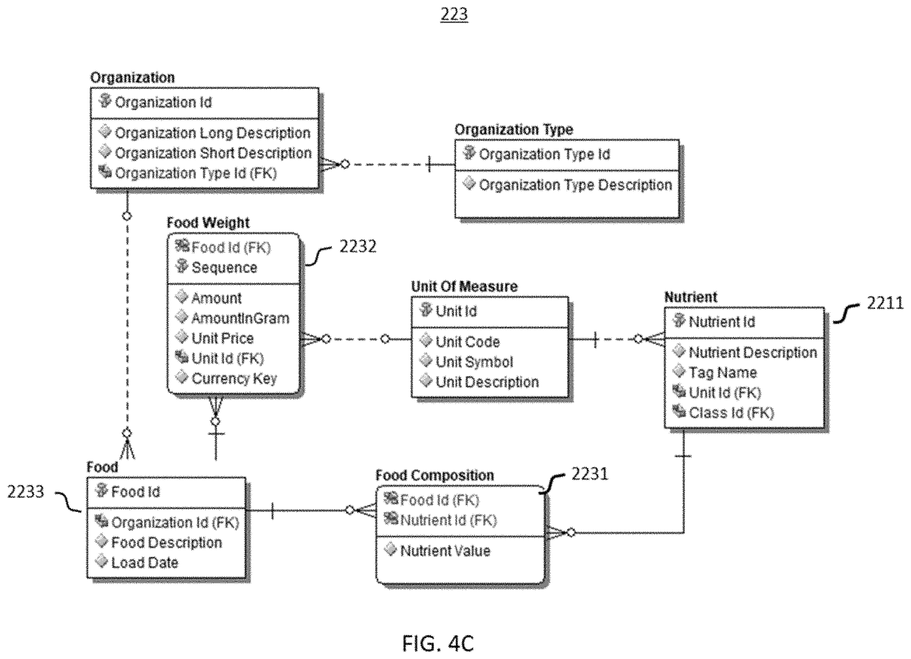

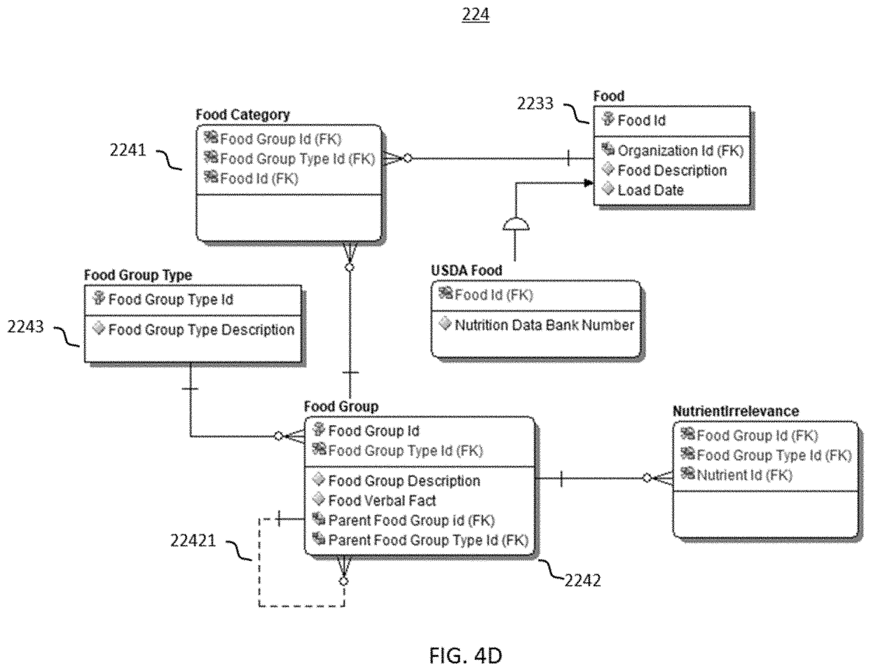

Various methods 500, 1300, 1400, 1500, 1520, 1530, 1700, 1800, and 1900 may be performed by way of associated computer-executable instructions according to some example embodiments. In various example embodiments, a memory device system (e.g., memory device system 130) is communicatively connected to a data processing device system (e.g., data processing device systems 110, otherwise stated herein as “e.g., 110”) and stores a program executable by the data processing device system to cause the data processing device system to execute various embodiments of methods 500, 1300, 1400, 1500, 1520, 1530, 1700, 1800, and 1900 via interaction with at least, for example, various databases 220, 221, 222, 223, 224, and 225 shown in FIG. 4. In these various embodiments, the program may include instructions configured to perform, or cause to be performed, various ones of the instructions associated with execution of various embodiments of methods 500, 1300, 1400, 1500, 1520, 1530, 1700, 1800, and 1900. In some embodiments, methods 500, 1300, 1400, 1500, 1520, 1530, 1700, 1800, and 1900 may include a subset of the associated blocks or additional blocks than those shown in FIGS. 7, 15, 15A-D, and 17-19. In some embodiments, methods 500, 1300, 1400, 1500, 1520, 1530, 1700, 1800, and 1900 may include a different sequence indicated between various ones of the associated blocks shown in FIGS. 7, 15, 15A-D, and 17-19.

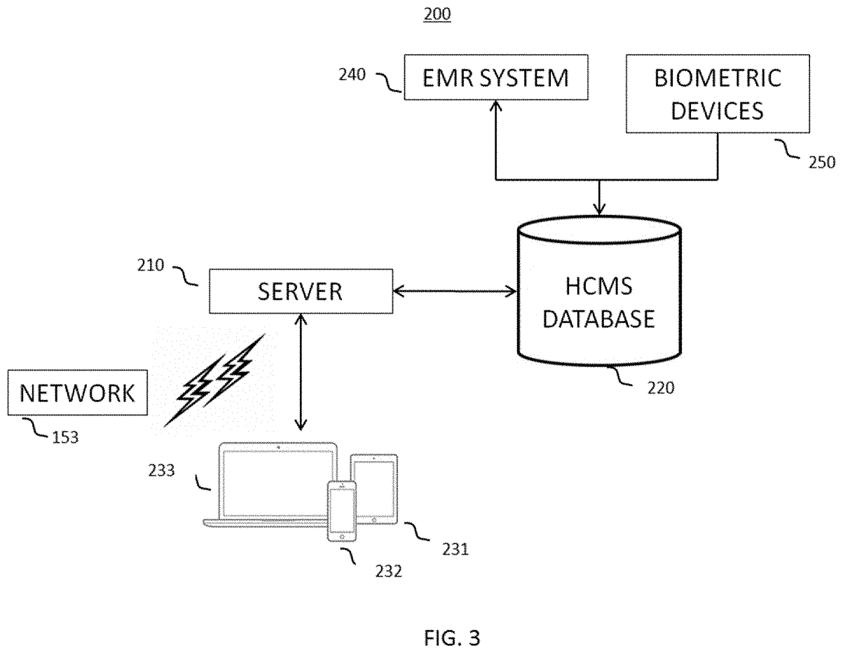

According to some embodiments of the present invention, the system 100 includes some or all of the HCMS system 200 shown in FIG. 3, or vice versa. In this regard, FIG. 3 illustrates a system 200, according to some embodiments of the present invention. The system 200 may be a particular implementation of the system 100, according to some embodiments. The system 200 may include wearable devices 250 and EMR system 240 that interact with a Health Cart management database 220 (also known as HCMS Database or GIAN Database). A user may access the HCMS 200 using one or more end-user devices 231, 232, 233. The end-user devices 231, 232, 233 include, for example, computers such as laptop, notebook, or desktop computers, or mobile devices such as cellular phones and tablets. The end-user devices 231, 232, 233 connect to a server 210 via a wired or a wireless network 153. In some embodiments, the server 210 includes one or more of a memory device system 130, data processing device system 110, and input-output device system 120 to execute the functions of the HCMS 200.

The HCMS system 200 accesses the data from the health cart management database 220 to evaluate, inform, and provide guidance to food shoppers regarding the “healthfulness” of the items they are putting into their shopping cart, either online or at the grocery store. The word “shopper” is substitutable with the word user or consumer. The word “healthfulness” is substitutable with the word healthiness or the phrases “high nutritional value,” “healthy health cart,” “healthy shopping cart” and the like. The phrases “shopping cart,” “grocery cart,” and “basket of foods” are all synonyms, aimed at referring to a collection of quantified food products. The adjective “quantified” means the quantity of each food item in the collection is known. Further, the word “meal” is a particular instance of a collection of foods and, therefore, can substitute the phrases grocery food and the like. It is to be understood that embodiments referring to grocery cart, shopping cart, or basket of foods are generally applicable to meal. The nuances behind meal and grocery cart are very minimal in the context of the HCMS system. Both are a collection of quantified food items. They may differ with respect to retention calculation: it is harder to estimate how long it takes for someone to consume a grocery cart item versus a meal because the latter is usually consumed within a day. The phrases “nutrient inadequacy” and “nutrient deficiency” are synonyms and refer to nutrients that are outside target, either in excess or in deficit, as compared to recommended targets. In some embodiments, alerts are provided when nutrient intakes are outside recommended targets. In some embodiments, “healthier” alternative food items are identified. In some embodiments, the HCMS system 200 generates an equivalent but healthier shopping cart by substituting one or more food items contributing negatively to one or more nutrients with a healthier alternative of the same type of food. In these embodiments, the phrase “shopping cart X is healthier than Y” means that the shopping cart X has a higher overall HC (Health Cart) score than Y. The phrase “food X is healthier than food Y” means that substituting Y with the same quantity of X in a shopping cart makes the overall HC score of the shopping cart higher. Further, the phrase “food X is healthier than food Y” along with “vis-à-vis N”, whereby N refers to a specific nutrient, means that substituting Y with the same quantity of X in a shopping cart makes the said nutrient N score higher. It is to be understood that the word “healthier” is relevant strictly in the context of a specific shopping cart whose food is consumed by a specific individual. Unlike the traditional food information given to shoppers, such as the nutrition facts label displayed on the back of every food package, specific to one product regardless of who is consuming it and regardless of what other products are consumed with it, the HCMS system provides personalized information taking into account medical and other conditions such as life style of individuals as well as the overall food items in the shopping cart. It is to be understood that the overall HC score behind the exact same shopping cart can be very different if consumed by individual X or Y. Further, the impact on the HC score of adding the same specific food item in two different shopping carts can be very different.

As shown in FIG. 4, the health cart management database 220 may obtain information from a variety of other databases. For example, in some embodiments, the health cart management system (HCMS) database 220 may receive or transmit information to one or more of a dietary reference intake sub model database 221, a nutrient coefficients sub model database 222, a food composition sub model database 223, or food taxonomy sub model database 224. In some embodiments, daily reference intake (DRI) tables 225 may be generated from the dietary reference intake sub model 221.

FIG. 4A shows an exemplar data structure for organizing information included in a dietary reference intake sub model database 221. The dietary reference intake sub model database 221 includes several tables such as dietary reference intake table 2210, nutrient table 2211, nutrient class table 2212, and unit of measure table 2213.

FIG. 4B shows an exemplar data structure for organizing information included in a nutrient coefficients sub model database 222. The nutrient coefficients sub model database 222 includes several tables such as nutrient table 2211, dietary disease coefficients table 2221, and diseases table 2222.

FIG. 4C shows an exemplar data structure for organizing information included in a food composition sub model database 223. The food composition sub model database 223 includes several tables such as food composition table 2231, food weight table 2232, and food table 2233.

FIG. 4D shows an exemplar data structure for organizing information included in a food taxonomy sub model database 224. The food taxonomy sub model database 224 includes several tables such as food category table 2241, food group table 2242, and food group type table 2243.

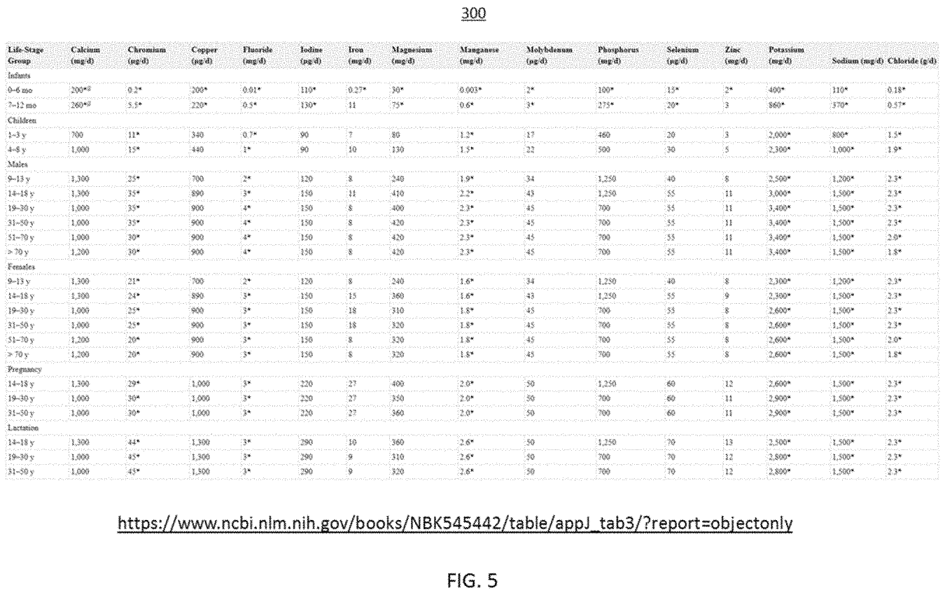

FIG. 5 shows an exemplar source dietary reference intake table 300 corresponding to a DRI table 225, according to some embodiments of the present invention. In some embodiments, the DRI table 225 may be predetermined and published by organizations such as the National Academy of Science, Engineering, and Medicine and WHO.

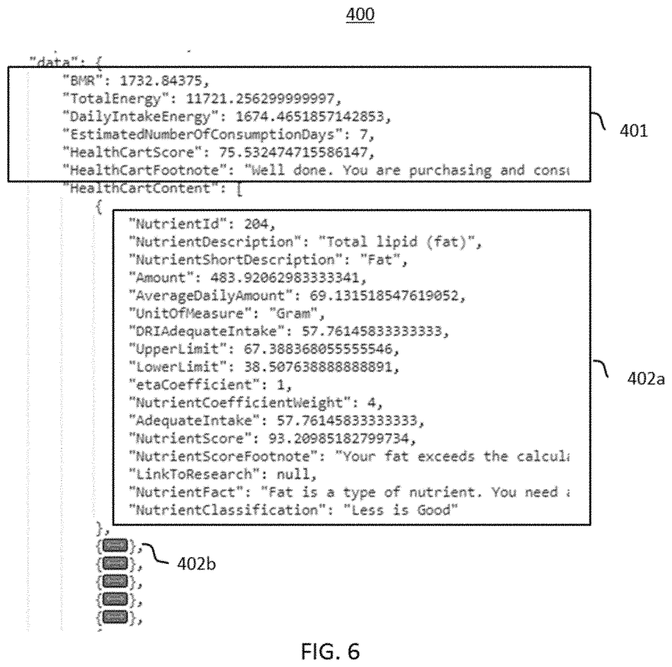

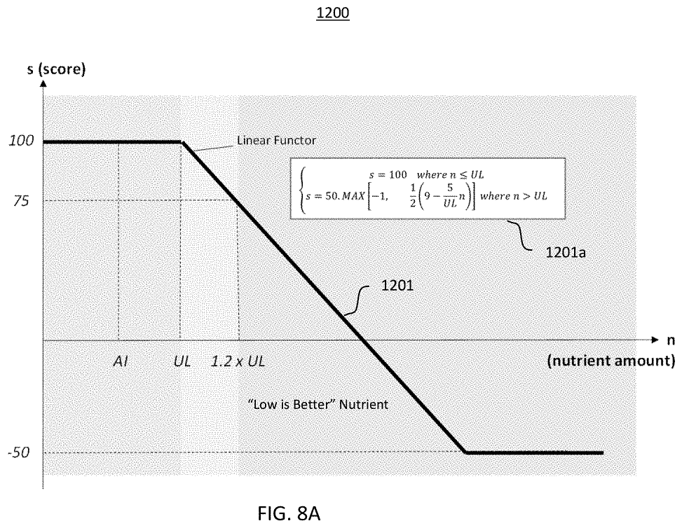

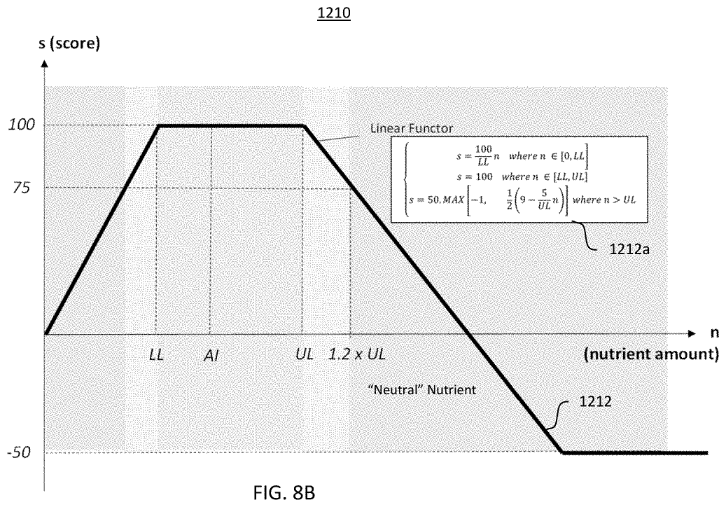

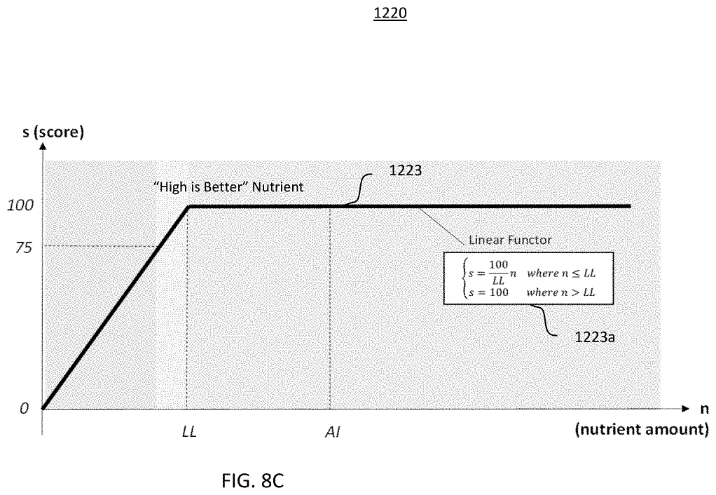

In some embodiments of the invention, a person's shopping cart is referred to as a Health Cart (HC), which summarizes the positives and negatives of the food put in the shopping cart. It is to be understood that, unlike a shopping cart, which is generic, a HC is personalized to a specific individual. FIG. 6 is an exemplar representation of a HC 400 generated by the method 500 shown in FIG. 7. FIG. 6 shows some key pieces of data conveyed by the HC among others. The overall structure is made of a header section 401 followed by several sections (402a, 402b) specific to each considered nutrient. FIG. 6 shows an exemplar section 402a (as a specific example of pone of the sections 402b) associated with a particular nutrient “lipid (fat).” The header section 401 contains, but is not limited to, the following pieces of data: 1) primary individual's BMR (calculated as described in later sections), 2) total energy in the underlying shopping cart (calculated as described in later sections), 3) average daily energy consumed (calculated as described in later sections), 4) estimated days for consuming the food (Total Energy/BMR), 5) overall HC score calculated in step 580 of method 500 shown in FIG. 7 and as described in later sections, and 6) footnote, which is a textual comment related to the overall score. Each nutrient section 402a, 402b includes, but is not limited to, 1) the nutrient internal identifier, 2) nutrient description, 3) short description, 4) total amount of that nutrient in the underlying shopping cart (calculated as described in later sections), 5) averaged daily amount calculated according to step 570 of the method 500 shown in FIG. 7 and as described in later section, 6) unit in which amount is expressed, 7) daily adequate intake (AI) coming from the table 2210, 8) an upper limit (UL) coming from the table 2210 or computed, 9) a lower limit (LL) coming from the table 2210 or computed, 10) η coefficient coming from system table 2221, 11) nutrient importance weight coming from system table 2221, 12) adequate intake (daily AI*η coefficient), 13) nutrient score calculated according to formulae such as 1201a in FIG. 8A, 1212a in FIG. 8B, or 1223a in FIG. 8C, 14) footnote related to nutrient score, 15) link to an external web site providing supplemental information, 15) nutrient fact coming from the HCMS system, or 16) nutrient class from table 2212. The assemblage of the pieces of data above embodying the HC is a compilation of informative nutrition facts specific to the shopper and the food in the shopping cart. Those nutrient facts, and other derivative pieces of data thereof, can be used to inform the shoppers, and their family members, of possible impacts, positive or negative, on their health. Further, they can be inputs or outputs to the methods, computation, methods and algorithms 500, 1300, 1400, 1500, 1520, 1530, 1700, 1800, 1900.

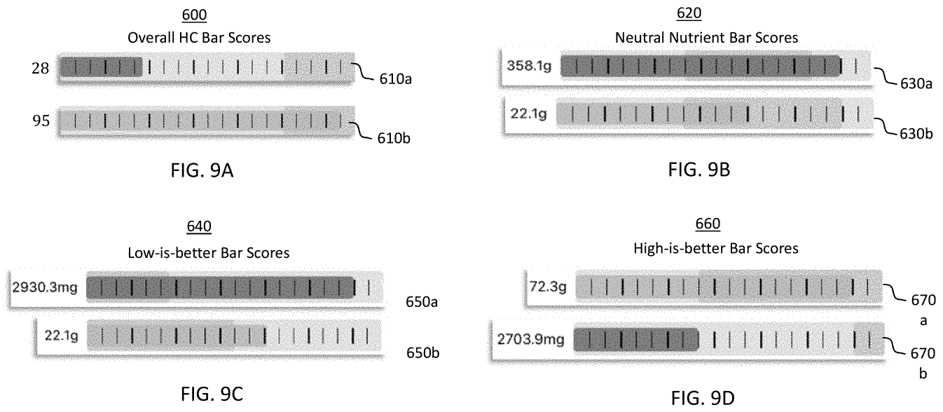

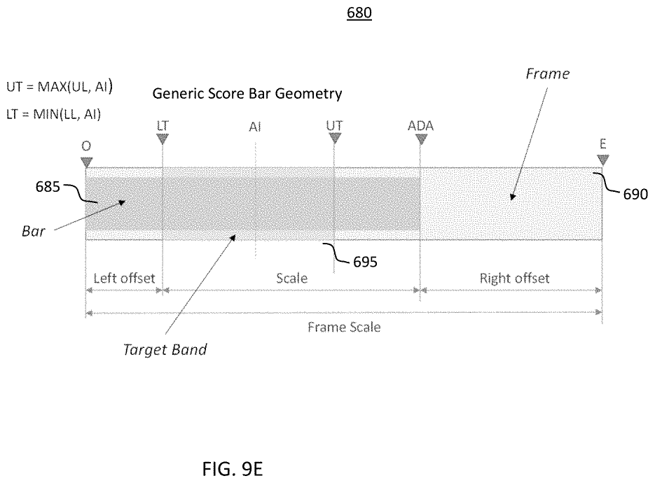

Graphical representations exemplar of an overall HC score 600 are shown in FIG. 9A. Bar 610a is a representation of a low score of 28 whereas bar 610b is a representation of a high score of 95. Graphical representations exemplar of different nutrient class scores 620, 640 and 660 are shown in FIG. 9B-D. Bar 630a represents an off-target neutral nutrient score, bar 630b represents an on-target neutral nutrient score. Bar 650a represents an off-target low-is-better nutrient score, bar 650b represents an on-target low-is-better nutrient score. Bar 670a represents an off-target high-is-better nutrient score, bar 670b represents an on-target high-is-better nutrient score.

FIG. 9E represents the seed geometry 680 of the exemplar score bars in FIGS. 9A-D. The components of score bar geometry 680 include the Frame 690, the Bar 685, and the Target Band 695. The length of the bar 685 is determined either by the HC score or the average daily amount (ADA). The position and the length of the Target Band 695 is determined by UT (Upper Target) and LT (Lower Target) values. The value of the Frame Scale is arbitrary defined and defines the geometric referential. The parameters are O (Origin), E (End), ADA (Average Daily Amount), LT (Lower Target), UT (upper Target), and AI (Adequate Intake). The values of these parameters are calculated given the average daily nutrient amount (ADNA) (or HC score), AI, LL, and UL and then mapped to pixels given a pixel referential defined by the Frame Scale (for instance 100 pixels).

FIG. 7 is a high-level flowchart of a method 500 to generate a HC according to some embodiments of the invention. The step 510 shows the two inputs of the algorithm: the shopping cart 510a (FIG. 7A) and the profile 510b (FIG. 7B) of the individual (shopper) that is consuming the whole, or a portion of, food in the shopping cart. Step 1500 is a sub-process executed by the method shown in FIG. 15 and calculates the retention of every item in the shopping cart. The step 520 adjusts the quantity of food consumed by the individual for which the HC is generated. For example, if some items are shared by the family, just a portion of the shared items will be consumed by the individual and that portion must be calculated given who is sharing the food and the individual family sharing factor (FSF). Then, for each considered nutrient N in the HC (YES to step 525), the step 550 is either getting the recommended target values (AI, LL, and UL) from the table 2210 (shown in FIG. 4A) or, if formulae exist, calculating them given arguments like the individual's basal metabolic rate (BMR). The next step 560 is applying some adjustment factors (η coefficients) to the targets, coming from the table 2221 (shown in FIG. 4B), in case the individual suffers from chronic diseases. The step 570 computes the estimated daily consumption of the considered nutrient N as described later and the step 580 computes the nutrient N score by comparing the nutrient daily consumption with the adjusted DRI targets determined in step 560 as shown with numeric examples later. If there is no more nutrient to consider in the HC (NO to step 525) then the next step 530 computes the overall HC score as the weighted mean of the nutrient scores calculated previously at step 580. The nutrient weight values are defined for each nutrient in the table 2221 (shown in FIG. 4B). Next step 540 is for gathering the multiple HC pieces of data calculated, or collected, along the different steps constituting the whole method 500 like an exemplar representation is shown in FIG. 6.

In some embodiments, the estimate of “daily consumption” is computed as a given food size divided by a period. The “period”, which is a time span expressed in number of days, is referred to “food item retention” or, in short, “retention”. The general definition of daily consumption applies to food as well as nutrient. In particular, the daily consumption of energy is estimated by dividing the total energy contained in a basket of food by the average retention of the items in it. The daily energy consumption contained in each food item is a key piece of information for calculating a HC; it allows the HCMS system to compute the total energy consumed daily by an individual. That value, expressed in kilo calories (kcal), is a match for the BMR value and, thereby, can serve, among other usage, to indicate whether an individual consumes too much food or not enough.

The BMR is a key piece of information that relates to the individual associated to the HC. As described above, BMR and food energy are intimately linked together; BMR can be related to food energy and vice-versa. In some embodiments, the HCMS utilizes that equivalence to compare daily food intake against recommended targets. The system relies on the commonly used Mifflin St Jeor's formula:

BMR (kcal/day)=10*weight (kg)+6.25*height (cm)−5*age (y)+s(kcal/day) (1)

where s is +5 for males and −161 for females

The formula (1) is supplemented in the system with a table of activity factors in order to take into account people's physical activities.

TABLE 1
Configuration Table: Activity Factors

| Activity Level | Factor |
|---|---|
| Sedentary | 1.2 |
| lightly active | 1.375 |
| moderately active | 1.55 |
| very active | 1.725 |
| extremely active | 1.9 |

Beside the Mifflin St Jeor's formula, the system can be configured to collect BMR coming directly from wearable devices 250 such as Fitbit and Apple Watch. Depending on the individual configuration, the system 200 can use either the formula or the BMR value received from the device(s).

The HC is configured with a predefined list of nutrients stored in the table 2211, constituting the considered nutrients, whose intake can be analyzed and compared to targets stored in the table 2210. The list of considered nutrients is a subset of the exhaustive list of food nutrients mapped out by the Institute of Medicine. Any particular instance of the system is configurable with an arbitrary subset of nutrients through a specific nutrition model.

Every nutrient falls in a classification as defined in the master table 2212: neutral, low-is-better, or high-is-better. Furthermore, the table 2210 shows that every nutrient is associated to a daily Adequate Intake (AI) representing the recommended daily ideal amount of a nutrient that should be consumed by anyone in good health. In supplement to the AI, a nutrient may be constrained by daily upper and/or lower limits (UL and LL). Each of the AI, LL and UL value is either universal, applicable to anyone, or personalized (variable for each individual), provided by tables or computed according to formulae. As an example of the latter, some macronutrient recommended intakes are proportional to the BMR (e.g. protein AI=0.15*BMR/4), dependent of the individual's physical age, measurement and gender. Another example is when the medical diagnosis (i.e. chronic diseases) of an individual requires to adjust the AI, LL and UL values using some η coefficients.

In some embodiments, the HCMS 200 may utilize data published by the National Academy of Sciences (US Institute of Medicine), but not limited to, with regards to the AI, LL, and UL values for the most common macro and micro nutrients. There exist several DRI tables 225, 300, such as one illustrated in FIG. 5, that provide daily reference values for each age group, sex, as well as pregnant women and whether or not they are suckling milk (lactation). These tables are the primary source of the system Dietary Reference Intake table 2210 in FIG. 4A, part of the system sub-model database 221. Importantly, note that the values in the DRI tables 300 represent references for healthy individuals. For that reason, the HCMS system 200 includes methods to adjust some reference values to take into account the constraints associated with certain chronic diseases. While AI values are generally specified by the Institute of Medicine the LL and UL target limits may be omitted in the DRI tables because they are unknown. The nutrient scoring algorithm, which leverages the invented instrument named herein “scoring functor” such as illustrated in FIG. 8A-C, can handle the case of missing targets. For some nutrients, the LL and UL can be calculated using specific formulae like the one for protein example used above. The system is configurable to include LL and UL computation any formulae if required.

The HC conveys a nutritional scoring process 580 in method 500 shown in FIG. 7 to compute a number inside an arbitrary range (e.g. between −50 and 100 but not necessarily constrained to those values), indicating by convention that −50 is the lowest score whereas 100 is the highest. Every nutrient is scored using a method based on the principle that the farther the nutrient amount is off the AI, or outside the target boundaries (UL or LL), the lower is the score. The scoring algorithm in step 580 takes into consideration the nutrient class 2212 to compute the score value because the computation is different.

The scoring algorithm utilizes a “scoring functor”1200, or simply “functor”, as shown in FIG. 8A-C. A functor such as 1200, 1210, or 1220 allows the system to calculate a nutrient score given, on one side, the Average Daily Nutrient Amount (ADNA), which is the estimated amount of a nutrient consumed daily, and, on the other side, the nutrient targets: AI, LL and UL. The shape of a functor reflects the risk on health of deficient nutrient intake plotted on a function curve. There are singular points (AI, LL and UL) on it, between which specific scoring functions apply. The curves show the score evolution given nutrient intakes. The higher is the score, the lower is the risk on health; a 100 score means a risk quasi null. The functors, and the scoring functions underlying them, vary according to the nutrient class. FIG. 8A-C depict three different functor morphologies, each associated to one of the nutrient classes. The functor 1201 applies to the nutrient of class low-is-better. The shape of the curve shows that if the nutrient amount is less than UL then the score value is the highest (e.g. 100). If the nutrient amount gets higher than UL then the score starts to decrease linearly down until the floor value representing the lowest score (e.g. −50) is reached. The functor 1212 applies to the nutrient of class: neutral. That class of nutrient (e.g., carbohydrate) is constrained by two boundaries; LL and UL. The score is the highest when the nutrient amount (n) is between the two limits. Outside that range the score starts to decrease down until 0 (score is 0 by convention) on the lower abscissa end and, on the higher abscissa end, until the score reaches the floor value, lowest score (e.g. −50). A singular instance of a functor of the neutral nutrient class can be reduced to a “caret” (\hat{ }) shape centered over AI in case both LL and UL limits are undefined. An example of a nutrient associated to a “caret” functor could be calcium, which is of class neutral with no boundary limits just constrained by an adequate intake (AI) value. The functor 1223 applies to the nutrient of class: high-is-better. The shape of the curve shows that if the nutrient amount (n) is more than UL then the score value is the highest (e.g. 100). If the nutrient amount gets lower than LL then the score starts to decrease linearly down to the score 0. The following section shows a few examples of nutrient score computation using the neutral functor (1212). The equations (1201a, 1202a, 1203a) of the functors 1201, 1212, 1223 are shown in FIG. 8A-C.

Some illustrative examples of score calculation done by the step 580 in the method 500 shown in FIG. 7 using the functors described in the previous section are shown below for the carbohydrate nutrient. In one embodiment, the daily carbohydrate limits are predetermined as LL=195 grams and UL=282 grams. Let's consider that carbohydrate is configured as a nutrient of class neutral in the system. A functor 1212 may be used therefore. If the daily carbohydrate intake in the HC is between that two values (e.g. Average Daily Nutrient Amount (ADNA)=200 grams) then the score is set to the maximum: s=100. If ADNA<195, ADNA=150 grams for instance, the value n=150 is plugged into the linear functor equation (1212a) where n is less than LL=195, yielding the score s=100/195*150=77. If ADNA>282, ADNA=450 grams for instance, the value n=450 is plugged into the linear functor equation (1212a) where n is greater than UL=282 yielding the score s=50*MAX (−1, (11−5*450/282)/8)=50*MAX(−1, 0.38)=50*0.38=19.

FIG. 8A-C depict a basic (linear) version of functors. In some embodiments, a quadratic version of functors may be used instead. The functions between the target points are portions of parabola instead of straight lines in a quadratic functor, but the nutrient scoring method stays essentially the same. The quadratic version is a mathematical sophistication to the linear functor that “smoothens” the score values, in particular the highest (e.g. 100) is granted in the case when the nutrient amount is matching strictly the AI value.

In some embodiments, the health cart includes a singular nutrient indicating the quantity of energy in food, expressed in kilo calories (kcal). That nutrient serves as summing up the entire energy conveyed by the HC, which represents the total energy of the food in the shopping cart as part of the header 401 in FIG. 6. As mentioned previously, the relationship between BMR and food energy is fundamental for assessing whether someone eats too much food. As a rough estimation, assuming the entire food in a shopping cart is consumed by one single individual, the average number of days, referred herein to average retention, can be estimated by dividing the total food energy by the BMR of the individual. Reciprocally, by the means of the same relation, the system is able to roughly estimate a person's adequate food quantity expressed in calories over a certain time span given his/her BMR.

The approximations made in the previous paragraph are quite rough. The HCMS 200 is based on a more precise, itemized, retention computation. FIG. 15 is a flowchart showing a method 1500 executed by the system 200 (and part of the method 500 shown in FIG. 7) to estimate the retention of each item in a shopping cart. Some food items in the shopping cart, like condiments, may be consumed over several weeks or months whereas others constituting, or part of, a meal like pizza, vegetable cans, etc. are likely consumed within a single day. That significant variation in consumption period is a reason why the retention of each food item must be evaluated separately in order to estimate more accurately the daily intake of every nutrient. The score of each nutrient depends strongly on the estimated food retention value: that is clear for instance that the overall impact on health of the nutrients contained in a 1 gallon bottle of soda is not the same if someone drinks one every day versus every week.

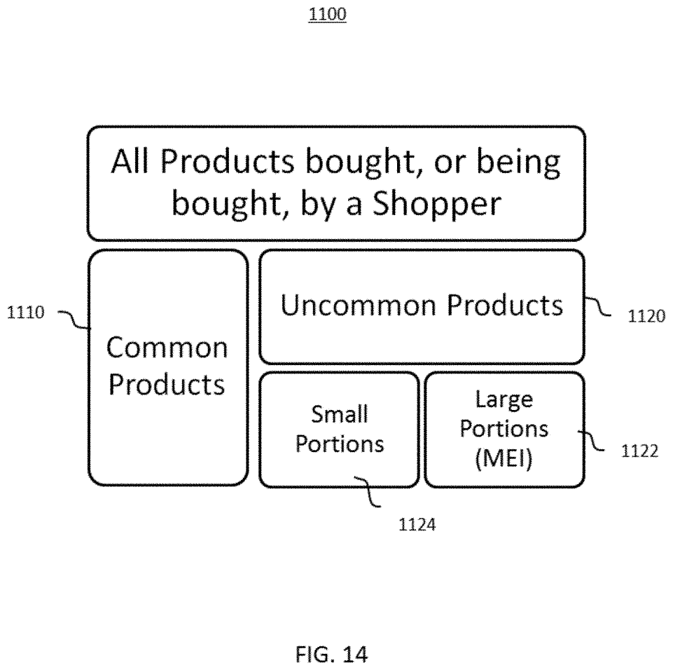

Accurate daily consumption estimate requires an appropriate item categorization because different methods of estimation exist but are only applicable to certain items whose specific shopping information is known. In some embodiments, the retention calculation depends on the categorization of the food items in a shopping cart such as depicted in FIG. 14.

The most accurate way to estimate food retention is, if available, to look back at shopper's purchase history. That is the reason why the HCMS system keeps track of every purchase and, therefore, is able to leverage data science and scholastic statistical methods to predict the period between two purchases of a given food item. In the method 1300 shown in FIG. 15C, the purchase history of an item F, or the same kind of F, is selected from the aggregate table 1310. The retention is assigned the purchase frequency in step 1320. All the items, or items similar to, for which there is sufficient purchase history, are designated as common products 1110 in FIG. 14 and their retention can be assessed with relatively high accuracy. The other items without, or lack of, history fall into the uncommon products category 1120. FIG. 14 shows furthermore that the uncommon products are broken further down into two subcategories 1122 and 1124:
1) The food items generally consumed regularly in small portions over a relatively long period. Condiments, snack containers, etc. fall typically into that category 1124.
2) The food items having large serving size (portion) such as meal or full meal cooking ingredients. Those items are referred as Mutually Exclusive Item (MEI) items 1122.

The MEI items are generally consumed within a day, a few days at the longest. That does not mean however that their retention is one or a few days. It can be much longer. Indeed, those food items are usually not all consumed within the same day. For instance, an individual, or an entire family, will unlikely be eating lasagna, spaghetti, pizza, and burger items within the same day, but will more likely eat them over a spread period of several days. Therefore the method to estimate the MEI retention in a shopping cart is trickier. The three categories of food: common items 1110, uncommon items 1120 with small serving size 1124, and uncommon items 1120 falling in MEI category 1122, each require a particular method to estimate their retention.

The method 1500 shown in FIG. 15 depicts an algorithm to estimate the retention of items in a shopping cart, according to some embodiments of the present invention. The methods 1300 and 1400 are two sub-processes permitting the method 1500 to calculate the retention of the common and uncommon products respectively. In step 1510, the BMR and the shopping cart (SC) are received. The computation algorithms of the underlying values: MEI Energy (step 1520) and residual BMR (rBMR) (step 1530) are shown in detail respectively in FIG. 15A (method 1520) and FIG. 15B (method 1530). The MEI energy is the summation of the energy of all the uncommon MEI items. The purpose of the MEI energy is to be evenly distributed across the uncommon items of large serving size, thereby allowing the HCMS system 200 to assess the average daily consumption of each.

FIG. 15A shows an exemplar method 1520 for calculating the MEI. In step 1521, the shopping cart (SC) is received. In step 1522, the MEI energy is initially set to 0. If there is an item F to be processed in the shopping cart (YES to step 1523), it is considered in step 1525 and if purchase history exists for that considered item F (YES to step 1526) then the item F is ignored and the method goes back to the step 1523. Otherwise, if the considered item F is uncommon without known history (NO to step 1526) then the method seeks the number S of servings per container at step 1527. If the serving size S is less than an arbitrary threshold (generally equating 4 but not necessarily that value), YES to step 1528, then the item F is of the category 1122: uncommon product with large portion, or in short MEI, as shown in FIG. 14 and the MEI Energy in the shopping cart is incremented with the total energy of the considered item F. To get the energy per 100 g of food the process 1529 is looking up the table 2231 in FIG. 4C given the food F identifier and the nutrient identifier referring to energy. If the number of servings of the item F is smaller than the threshold (NO to step 1528) then the item is ignored and the method 1520 passes to the next item in the shopping cart at step 1523. If all the items have been processed (NO to step 1523) then the method returns the value stored in MEI Energy.

Since this is an averaged value, every MEI item in a shopping cart has the same retention, called Default Retention calculated at step 1540 as the MEI Energy divided by the residual BMR. It yields naturally a number of days since that is the ratio of kilo calories over kilo calories/day. The residual BMR calculated at step 1530 in the method 1500 and shown in detail in FIG. 15B is the portion of the shopper's BMR left after removing the daily energy of the common and the small portion uncommon products for which the retention is determined individually using different methods.

FIG. 15B shows an exemplar method 1530 for calculating the residual BMR. In step 1531, the method receives the two arguments: the shopping cart SC and the BMR of the user. The variable EDCE (Every Day Consumed Energy) is initialized to 0 in step 1532. If there is an item F to be processed in the shopping cart (YES to step 1533), it is then considered in step 1534. The next step 1535 computes the total energy contained in the item F and stores the value in the variable E. If purchase history exists for that considered item F (YES to step 1536) then the average purchase history is selected in step 1320 from the table 1310 and temporarily stored in the variable T. The step 1537 increments then the EDCE value to the daily energy consumption of the item F, calculated as the total energy E divided by the retention T. If, the considered item F has no purchase history (NO to step 1536) then the step 1420 seeks the number S of servings per container. If the serving size S is less than an arbitrary threshold (generally equating 4 but not necessarily that value), YES to step 1538, then the item F is of the category 1122: uncommon product with large portion, or in short MEI, as shown in FIG. 14 and ECDE is incremented with the total energy E calculated previously in step 1535 divided by the number of servings S in step 1539. If the number of servings of the item F is smaller than the threshold (NO to step 1538) then the item is ignored and the method 1530 passes to the next item in the shopping cart at step 1533. If all the items have been processed (NO to step 1533) then the final step 1560 of the method 1530 returns the difference between the user's BMR and the ECDE value, which is the definition of the residual BMR (rBMR) of a shopping cart.

After the default retention DR is calculated in step 1540 the retention is determined for each consumed item in the shopping cart (YES to step 1545). Then, in step 1550, the method considers every shopping cart item one by one. The step 1555 determines whether the item, or a similar one, has purchase history. If history does exist then the sub-process 1300 is triggered, otherwise the sub-process 1400 is triggered instead. And that process keeps going until the retention of all the SC items are processed (NO to step 1545) and the method 1500 stops. The sub-process method 1300 shown in FIG. 15C processes the common products for which history exists. It receives an item F at step 1305 and, in step 1320, obtains the purchase frequency, which is averaged in the table 1310 given the past item purchases, or those from an item of the same kind, The step 1330 records the item retention as the average purchase history obtained in step 1320. The sub-process method 1400 shown in FIG. 15D processes the uncommon shopping cart items for which no history is available. The step 1405 receives an item F and the default retention DR calculated in the parent method in step 1540. The step 1420 seeks the number of servings per container of the item F, which is recorded as a specific unit of measure in the table Food Weight 2232. If the item has large servings (NO to step 1425), that is classified as large portion (MEI) in the categorization in FIG. 14, then the item retention is set to the number of servings in step 1430. If the servings per container is small (YES to step 1425) then the item retention is set to the default value DR computed in step 1440. It is to be understood that assigning an item retention to the number of servings per container as performed by the method 1400 in FIG. 15D is an assumption made on the premise that the shopper, and/or the household, is consuming the food reasonably, in other words, the daily consumption must be near the portion defined by the servings per container. That optimistic, but uncertain, assumption is the reason why the retention of the common items, for which the average purchase frequency is very much known, is generally more accurate. By acquiring or, keeping track of past purchases, the HCMS 200 is learning the consumer's shopping pattern over time and, therefore, estimates the food item retention more and more accurately.

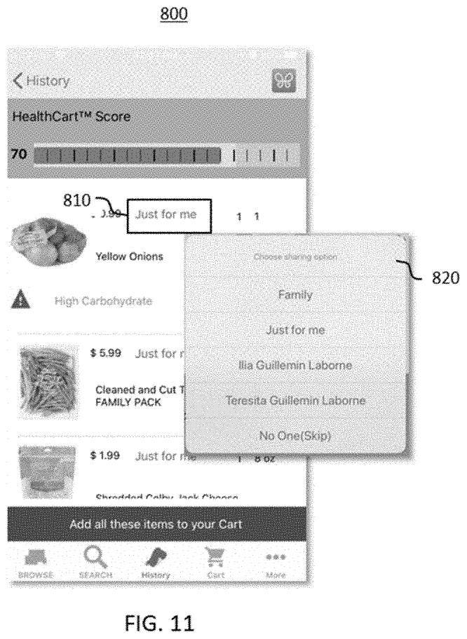

The food in a grocery cart is not always consumed by the shopper alone. Some items in a shopping cart can be consumed by the entire family or even by external guests outside the household. FIG. 11 shows a possible user interface that permits a user to specify whether each shopping cart item is either shared by the family, consumed by the shopper alone, consumed by one or several specific member(s) of the family, or otherwise may be ignored for scoring the HC. Sharing selection 810 is a sharing option that can be selected from a sharing pop-up menu 820. In that particular configuration, the user can share the item with its entire family, just for him or specific member(s) of the household, or be skipped from the HC scoring (“No One” option).

If an item is shared across the family (sharing option=Family), the portion eaten by each family member needs to be assessed in order to compute the daily consumption. In some embodiments, the HCMS system leverages the Family Sharing Factor (FSF) to adjust the quantity of food consumed daily by each member prior to compute the HC scores; this adjustment is performed by the step 520 in the method 500 shown in FIG. 7. In step 520, the FSF is computed given the BMRs of every family member. By definition, the FSF of each family member is computed as the ratio: member's BMR divided by the sum of the family's BMRs. For example, let's assume a parental family with one child for which the father's BMR is 2,800, the mother's BMR is 1,700 and the child's BMR is 1,100. The overall BMR of the family is 2,800+1,700+1,200=5,700 kcal. According to that definition, the FSF value of the three members is respectively: 2,800/5,700=0.49, 1,700/5,700=0.30 and 1,200/5,700=0.21. The appropriate factor will be applied to the quantity of every shopping cart item shared by the family before computing the HC of a given family member. For example, if a 3 lb. package of apples is shared by every family member then the respective FSF factors will be applied so that the father will be assumed to consume: 3×0.49=1.47 lb., the mother: 3×0.3=0.9 lb. and the child: 3×0.21=0.63 lb. of apples. If an item is not shared by every family member but just by some then the FSF of each consumer of that item must be augmented by a factor reflecting the bigger portion of food consumed. That factor is 1/sum (consumers' FSF). For instance, if the father is not eating apples then the mother and the child will eat more by a factor of 1/(0.3+0.21)=1.96. The respective quantity of apples consumed by the mother and the child is thereby 3×0.3×1.96=1.76 lb. and 3×0.21×1.96=1.24 lb. respectively.

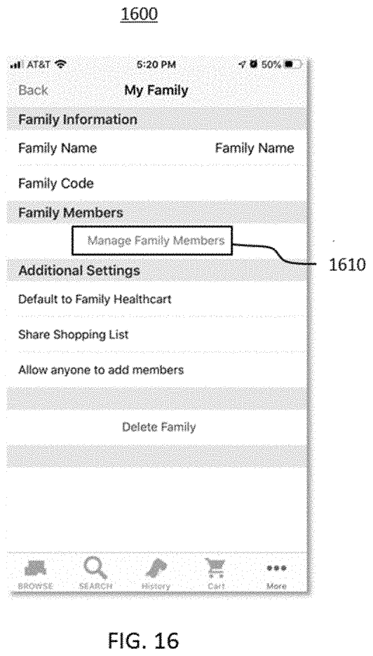

In some embodiments the family members can be added into the HCMS system by the primary shopper by updating his/her user profile. The HCMS allows the head of the household, defined as the primary shopper, to add family members using user interfaces such as the exemplar interface 1600 shown in FIG. 16. The actionable link 1610 allows the primary shopper to add members to his/her family.

The previous sections cover the possible gap when food is bought but not fully consumed by the primary shopper or the household. Conversely, consumed food can be eluded because it is not tracked by the HCMS system 200. There is a common situation where, for example, people eat outside home, at work place or in restaurants for instance. Another situation is when a household cultivates its own produce and, therefore, does not shop for those items. These cases are handled by allowing the HCMS users to update their user profile by indicating how many meals a week they assess being excluded from their grocery shopping. They can also specify an overall percent of food they believe being consumed but not tracked by the HCMS over an average period of a week for instance. A value of 40% would mean for instance that the user considers that 40% of what he/she consumes is not coming from grocery purchases. The quantification of that gap allows the HCMS to adjust appropriately the retention in order to reflect it on the daily consumed quantities, which is virtually more than what is contained physically in the shopping carts managed in the system.

It is to be understood that the daily nutrient recommended values (AI, LL, and UL) are not always the same with regards to people's health. For instance, an overweight person that desires to lose weight may have his/her carbohydrate targets reduced by 250 kilocalories a day as compared to the usual dietary recommendation (the daily AI of carbohydrate in grams is set by reference to 0.55*BMR/4). The table 2221, part of the HCMS database 220, allows the HCMS system 200 to configure the adjustments to make on specific nutrient targets with regard to a chronic disease. An adjustment is made by the means of a multiplying factor, named eta-coefficient (written 11 coefficient), that is set to be applied against the reference values: adequate intake and/or target limits. The default η coefficient value is set to one; a multiplying factor of one is neutral, not modifying the reference value. A η coefficient value greater than one is set to raise a reference value whereas a η coefficient value less than one is set to lower a reference value. For instance, it would be appropriate to set the carbohydrate η coefficient to 0.875 in order to lower the target values of people suffering from obesity by around 250 kilocalories, based on the arbitrary assumptions that 1) the carbohydrate target is proportional to BMR and 2) that the population BMR is 2,000 kilo calories on average (1−250/2,000=0.875). The system table 2221 stores the η coefficient values. Table 2221 is related to the table 2222 as shown in FIG. 4B. Table 2222 hosts the disease master such as commonly defined in the Electronic Medical Record (EMR) systems. The table 2221 includes the ICD10 code (International Classification of Diseases, Tenth Revision) column, which permits the assignment of a specific η coefficient value to a disease. The η coefficient values are stored in the column: Eta Coefficient, for each combination of a nutrient (column: Nutrient Id) and a chronic disease (column: ICD-10-CM Code).

TABLE 2
Exemplar content of the table 2221 Dietary Disease Coefficient

| Coefficient | Validity | Nutrient | ICD-10-CM | | Eta | Nutrient | Validity |
|---|---|---|---|---|---|---|---|
| Id | Start Date | Id | Code | | Coefficient | Weight | End Date |
| 1 | Feb. 05, 2019 | 1 (Carb) | E66 | (Obesity) | 0.875 | 5 | Unknown |
| 2 | Feb. 05, 2019 | 1 (Carb) | E08 | (Diabetes) | 1 | 3.5 | Unknown |

In some embodiments, the η coefficient values are set to one in the system table 2221. Only the rows referring to a chronic disease requiring changes to the usual dietary reference value of a certain nutrient have different η coefficient values. The specific η coefficient value of each considered nutrient is included into the HC as shown inside the 402a section in FIG. 6, essentially to adjust the recommended AI, LL and UL values, as processed by the step 560 in the method 500 shown in FIG. 7.

In some embodiments, the HCMS 200 is based on the food nutrient composition providing the ability to compute nutrient intake like in the step 570 of method 500 shown in FIG. 7. Every food item is composed of nutrient amounts that are recorded in the table Food Composition 2231 in FIG. 4C. Every considered nutrient present in the food item is stored as a specific row in the Table 4 below, in which the nutrient amount (column: [Nutrient Value]) is stored, expressed in the weight unit of measure associated to the nutrient (refer to table: Nutrient 2211). By convention in that document, the nutrient value is the weight expressed in grams relative to 100 grams of food. In conjunction with the Food Composition table 2231, the table: Food Weight 2232 allows the process 570 to compute the overall nutrient content given the food weight.

TABLE 3
exemplar content of the table: Food Weight 2232

| | | | Amount | Unit | | | |
|---|---|---|---|---|---|---|---|
| Food Id | Seq. | Amount | In Grams | Price | Unit Id | | Currency |
| 1 (Eggs) | 1 | 1 | 672 | $2.00 | 14 | (Container) | 1 (US $) |
| 1 (Eggs) | 2 | 1 | 58 | $0.21 | 3 | (Large egg) | 1 (US $) |

TABLE 4
exemplar content of the table: Food Composition 2231

| Food Id | Nutrient Id | Nutrient Value |
|---|---|---|
| 1 (Eggs) | 2 (Protein) | 12.56 |
| 1 (Eggs) | 3 (Fat) | 9.51 |

For instance, given a container of 12 fresh eggs weighing 672 grams according to the exemplar Table 3 above and given the protein amount in it is 12.56 grams (per 100 grams) according to the exemplar Table 4 then the overall protein amount in the container can be calculated as: 12.56*672/100=84.4032 grams.

The food item retention calculated according to the method 1500 permits the step 570 in the method 500 in FIG. 7 to estimate the daily intake of every nutrient in a HC. Using the same example of protein in eggs as above, assuming that a shopper is buying a container of 12 fresh eggs every week, that is a retention of 7 days, then the estimated protein intake will be 84.4032/7=12 grams per day. Adding up the protein intake of every other shopping cart item the same way yields the total average daily intake of protein. That total intake value is then a match for the protein recommended values: AI, LL and UL. The step 580 in the method 500 is thereby able to determine whether the protein intake is between the recommended or off targets and calculates accordingly the protein score according to the formulae shown in FIGS. 8A-C. That method works naturally the same way for any considered nutrient within the nutrition model underlying the HC.

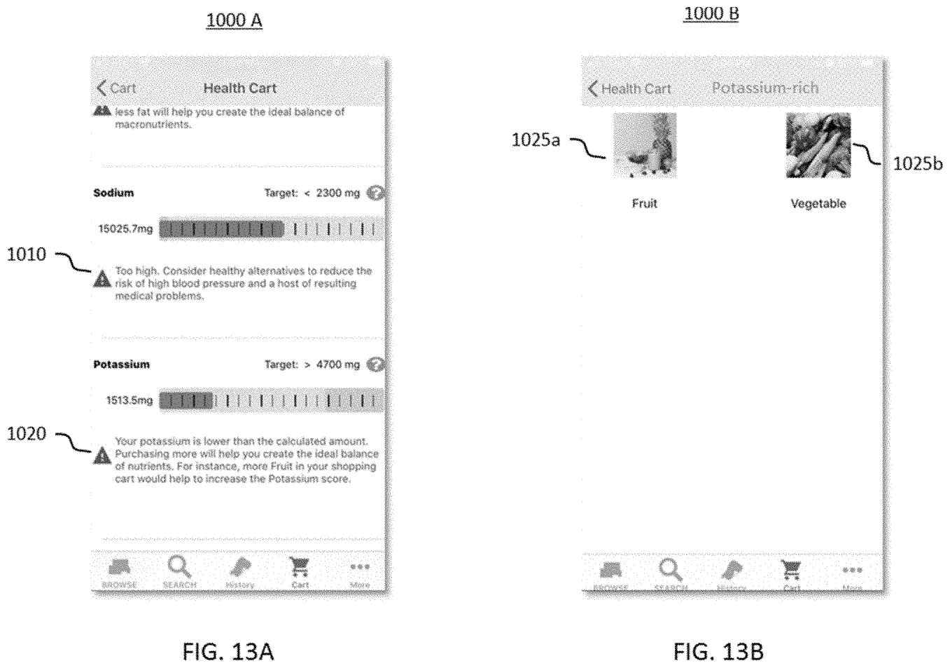

In some embodiments of the invention, there is a method to identify a food item in a shopping cart that contributes to make considered nutrients off the desired targets. Such an identified item is said “nudged” in the system. FIGS. 13A and 13B show examples of shopping cart items that are nudged; an exemplar user interface 1000A shows nudges for two nutrients Sodium and Potassium. The first step of the nudging process, performed as part of the step 540 in the method 500 shown in FIG. 7, is to identify every nutrient that is off target in the HC. That is done by looking up the nutrients whose score is less than the maximum value (e.g. 100), or lower than an arbitrary threshold (e.g. 80% of highest score). Those identified nutrients are designated as deficient nutrients. If a nutrient is deficient in the HC that is surely due to one or several shopping cart items, which contain either too much, or not enough, amount of it. In the case of having too much of a nutrient, the HCMS system 200 can sort descending the shopping cart items by their individual contribution to the overall nutrient content. The top ones identify clearly the food items that impact negatively the most a nutrient. In the case of not having enough of a nutrient, just sorting ascending the items by the content does not necessarily indicate that the top ones must be nudged. Some nutrients, such as vitamins or minerals for instance, are never present in some kind of food. Such nutrients should not be the reason for nudging that kind of food. In that case, the way to higher the nutrient amount is rather to add an item of another sort to the shopping cart. For example, it would not be appropriate to nudge chicken like “low in fiber” because meat in general does not carry fiber. The food items low for a certain nutrient are nudged only if alternative items exist in the same food category. For instance, the addition of fruits such as pears or apples in the shopping cart would be a better way to higher the fiber amount rather than attempting to seek a meat alternative.

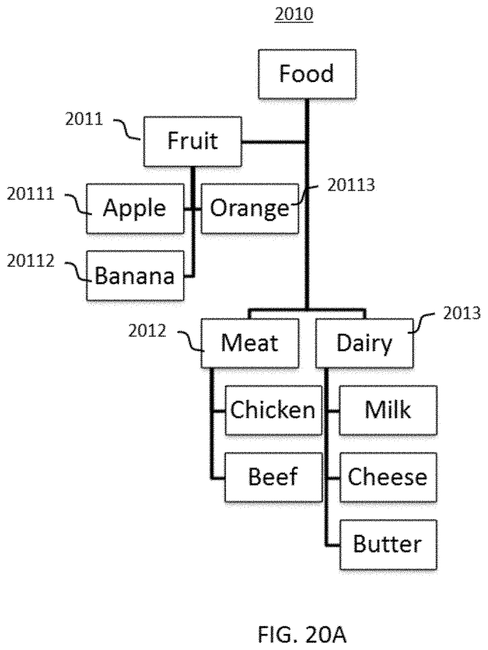

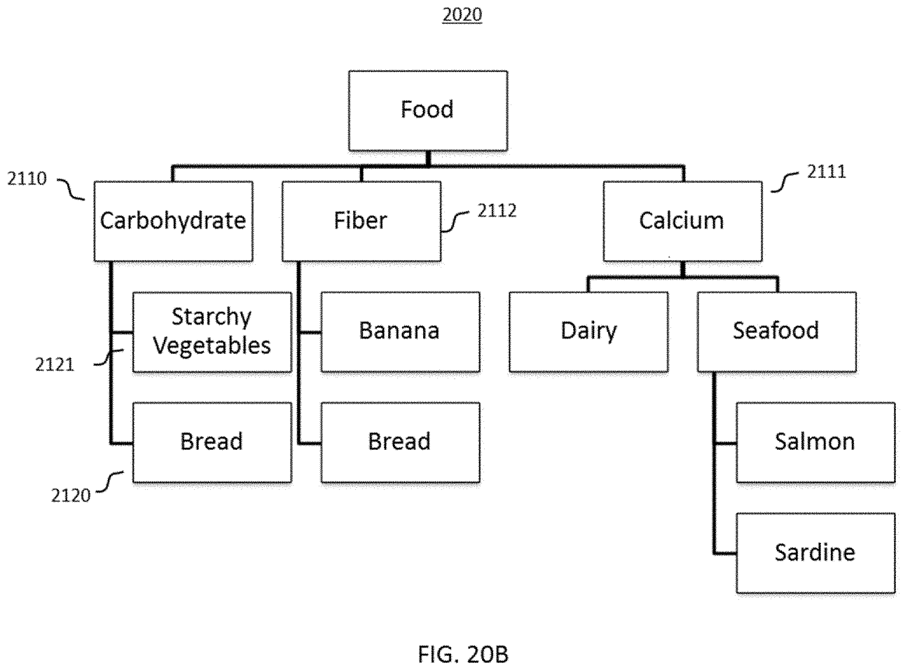

In some embodiments, there is a method provided to analyze the scores of every nutrient in the HC. The weak nutrient scores, under an arbitrary threshold, allows the HCMS system 200 to identify the inadequate nutrients in the HC. The nutrient inadequacy comes from an inappropriate daily intake, either in excess, in the case of neutral or low-is-better nutrients, or in deficit, in case of neutral or high-is-better nutrients. The icon 1010 in the FIG. 13A is an exemplar representation of neutral or low-is-better nutrients in excess. The icon 1020 in the FIG. 13A is an exemplar representation of neutral or high-is-better nutrients in deficit. Activating the icon 1010 will list the food items in the shopping cart contributing the most to the content of the nutrient in excess. If the user touches or clicks the icon 1020 then a list of food categories 1025a and 1025b rich in the selected nutrient in deficit (potassium is the considered example) is displayed in the user interface 1000B as shown in FIG. 13B. The user can then select a food category to get a list of food rich in the selected nutrient. The list of food categories is determined by utilizing a specific food taxonomy configured in the HCMS whose exemplar is sketched in the FIG. 20B; the list of food categories is placed under a parent node representing a nutrient. The exemplar food taxonomy 2020 shows that, for instance, starchy vegetables 2121 and bread 2120 are two categories rich in carbohydrate 2110. Like any food taxonomy utilized by the HCMS system 200, the food taxonomy 2020 is configured in the tables 2242 in the FIG. 4D.

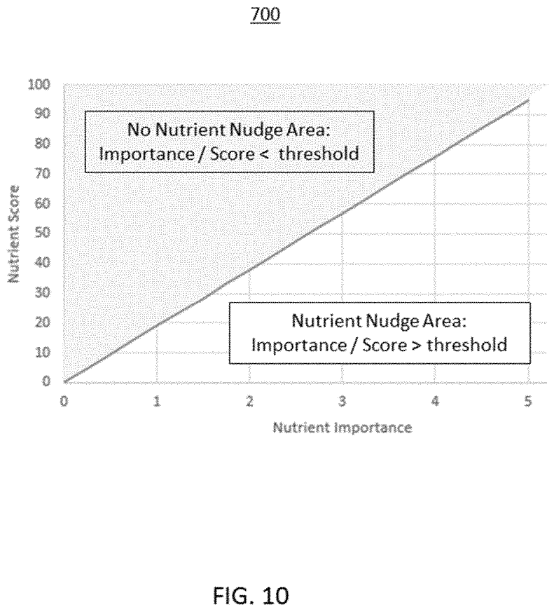

It is to be understood that nutrients do not have always the same impact, or importance, with regards to people's health. For instance, a high sugar level can be critical for diabetics while less impacting a healthy active person. The HCMS system 200 is designed to configure the importance of each nutrient with regard to a given chronic disease. The step 540 in the method 500 shown in FIG. 7 utilizes the nutrient importance to decide whether a food item must be nudged or not depending in which of the two areas illustrated in FIG. 10 the nutrient falls into. If the ratio of importance to score is less than a threshold, no nudge is performed. If the ratio of importance to score is greater than the threshold, the nudge process is performed. The system table 2221 in FIG. 4B stores the nutrient weight value that defines the importance: the higher is the value, the more important is the nutrient. In some embodiments, the system table 2221:

Dietary Disease Coefficient hosts a weight value for each considered nutrient and each disease identified with an ICD10 code. The unusual, but valid, value=0 means that the nutrient is considered irrelevant in the system.

The sum of the nutrient weight values must equate the number of considered nutrients in the HC. That is because the nutrient weights are also the ponderation coefficients in the weighted mean that yields the overall HC score such as computed in the step 530 in the method 500 shown in FIG. 7. That constraint must be satisfied in the system when initially configuring the nutrient weights for every chronic disease as well as when calculating the comorbidity nutrient importance values.

The table 2221 is related to the table 2222 in FIG. 4B. The latter is hosting the disease master such as commonly defined in the Electronic Medical Record (EMR) systems. The table 2221 includes an ICD10 code (International Classification of Diseases, Tenth Revision) column, which permits assigning an importance weight to a specific disease. So that every nutrient has a weight value that quantifies its importance in the HC. Further, every nutrient has a score that reflects how far the nutrient is off the recommended target in the HC. In some embodiments, the value of the ratio Importance Weight/Score Value is suited to determine whether a nutrient may be nudged or not. As shown in FIG. 10, an important nutrient with a low score, therefore having a high ratio, may be nudged whereas a less important nutrient with an upper-range score, therefore having a low ratio, does not need to be nudged. There exists a predetermined configurable threshold value (e.g. 1/19=0.0526) over which the nutrient will be nudged. FIG. 10 shows the two areas, nudging area versus not nudging area, on a scatter graph whose ordinate represents the score value defined on the domain [0, 100] and the abscissa represents the importance weight defined on the domain [0, 5]. These axis domains are arbitrary and illustrative in FIG. 10.

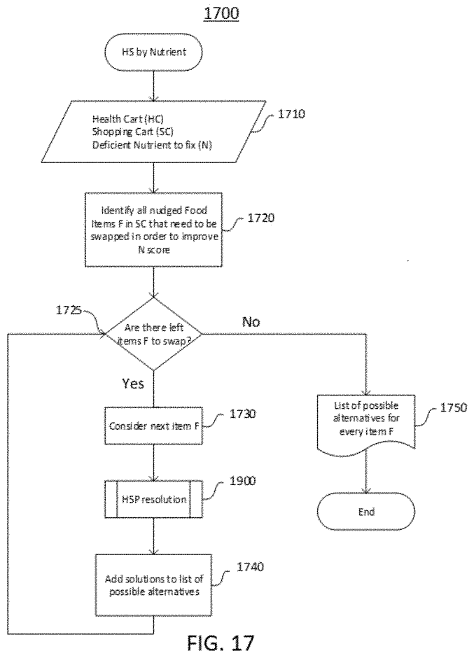

The food nudging method described above is able to sort the alternative items by impact on a given nutrient deficiency. In the case of having too much of a nutrient, the system restricts the nudged items to those possessing alternatives having a significant impact on the nutrient improvement. The impact significance is quantified in the system by the means of a percentage (W %) that involves the gain of the alternative item relatively to the total nutrient amount, noted W. The gain G is the difference of amount between the original wₒ and alternative wₐ amount: G=wₒ−wₐ, so that W %=G/W. Furthermore, an Average Contributing Factor (ACF) is introduced as 1/N, where N is the number of items in the shopping cart, in order to take into account the relative contribution of each item to the total nutrient amount in the HC. For instance, if a shopping cart is filled in with 10 items then the ACF is 0.1, endorsing thereby a 10% average contribution of every item on the HC nutrients. A threshold T can then be set to an arbitrarily percent value (e.g. 20%) so that only the alternative items satisfying the inequality: W %>ACF (1+T) are retained as having a significant impact, nudged therefore by the system with regards to a given nutrient. The nudging determination is followed by the step 1720 in the method 1700 shown in FIG. 17 for determining whether an item must be considered to get swapped.

A method to compute the adjustment factors to apply in case when an individual suffers from comorbidity, several chronic diseases simultaneously, is described next. This method is utilized by the step 560 of the method 500 shown in FIG. 7 in order to adjust the recommended daily intake values. In some embodiments, the η coefficients are adjustment factors to nutrient targets in regards to specific diseases. Beside the η coefficients, the nutrient weights rank the nutrient importance vis-à-vis one specific disease as described above. Both coefficient and weight values are configured in the system table 2210 in FIG. 4B tied to one single chronic disease. In case of comorbidity, the η coefficient and importance weight values of every disease must be merged. In some embodiments, the following comorbidity merging rules are applied: 1) the comorbidity η coefficient is the lesser among every considered disease if every value is less or equal to one, otherwise the greater η coefficient if every value is greater or equal to one, 2) the comorbidity importance weight is the higher weight among every considered disease. The higher importance always prevails on the lower one in rule (2) so that the sum of the weights can be greater than the number of considered nutrients, based on the defined constraint. That is resolved by applying the factor N/N_{co} to every importance weight, N being the number of considered nutrients and N_{co} the sum of the adjusted weight values after having applied the rule (2). It has to be noticed that the rule (1) can create some conflicts; if one disease has a η coefficient greater than one and another one less than one then the rule (1) is inapplicable. The system alerts the user if that case is happening and the η coefficient is set to one by default.

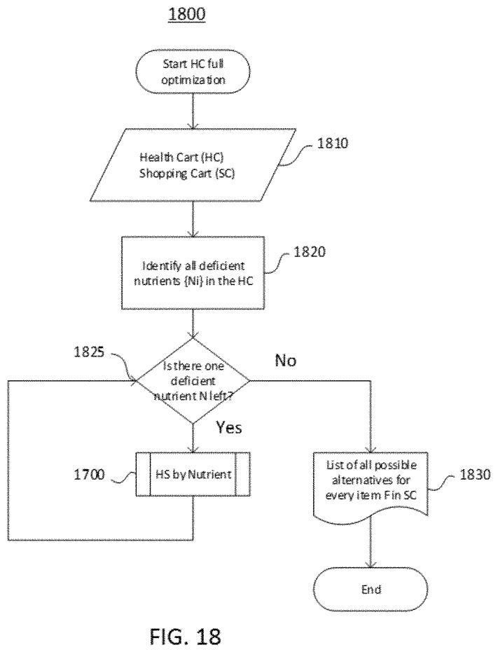

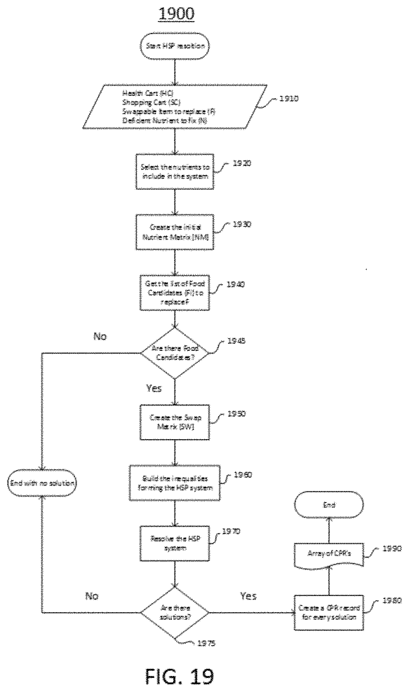

There is described a method 1900 to seek substitutes when a food item is nudged in a health cart. A substitute is a food item of the same sort that will make the total daily amount of a nutrient closer to, or within, target. The method 1900 is referred as Healthy Swap (HS) and shown in FIG. 19. Further, there are two extended HS methods 1700 shown in FIGS. 17 and 1800 shown in FIG. 18. The fundamental healthy swap method 1900 describes a basic HS method to substitute a single item aimed at improving one single nutrient; the second healthy swap method 1700 describes a generalized HS method aimed at replacing, at once, every shopping cart item contributing negatively to a single nutrient; and the third healthy swap method 1800 describes the ultimate overarching HS method aimed at replacing every shopping cart item contributing to any of the deficient nutrients in a HC. The HS methods 1700, 1800, and 1900 require the resolution of a non-trivial problem referred as the Healthy Swap Problem (HSP). That is a kind of problem classified as operations research dealing with optimization given constraints, in the case of the HCMS system 200, over Boolean variables. Because that is a puzzling problem, the method of resolution starts with the formalization of the problem. The goal is to construct a system of inequalities whose solution(s) yield(s) a food item healthier than the original one. In that specific context, a food item is considered healthier if at least one of its nutrient quantity is moved either under target (between a lower and upper value) or, if not possible, has reduced the gap. In order to prevent other nutrient degradation after the substitution, the method takes into account several nutrients. It is still focusing on improving one, ensuring nonetheless that the others either stay between targets or get closer to the targets.

In some embodiments, the HSP problem can be construed to a multi-variable system of equations, mainly inequalities. It is expected that this system is solvable by the mean of usual system resolution algorithms. In some embodiments, the equations behind the HSP system are made of Boolean variables, which greatly facilitate the resolution. Further down, some numeric examples show how to find solutions to the HSP system when they exist. Finally, the generalization of these examples allows streamlining the foundation of the HSP algorithm described by the method 1900 shown in FIG. 19.

A basket of food is a set F of food items inside a shopping cart or part of a meal. The set F is reduced to two elements: T and S₀, denoted as:

F={T,S₀},

where T represents the summation of every food item, S₀ representing the swappable item targeted to be replaced. It is to be understood that, according to that definition, F contains always 2 elements, nonetheless with no limit of the total number of food items in the basket.

Both elements, T and S S₀, convey their own nutrient quantities that can be organized like in the table below.

TABLE 5
tabular representation of the initial state of the HSP problem

| | n₁ | n₂ | . . . | nₚ |
|---|---|---|---|---|
| T | T₁ | T₂ | | Tₚ |
| S₀ | q₁ | q₂ | | qₚ |
| T − S₀ | Q₁ = T₁ − q₁ | Q₂ = T₂ − q₂ | | Qₚ = Tₚ = qₚ |

In Table 5, the Tⱼ terms denote the overall nutrient quantities of the whole set whereas the qⱼ terms are the nutrient quantities of the swappable item. The highlighted cells represent a 2×p matrix denoted Nutrient Matrix [NM].

The [NM] cell values set the initial state of F before the food S₀ is substituted. Qⱼ is the difference: Qⱼ=T₀ⱼ−qⱼ, which is the total nutrient quantity of the whole initial set apart the swappable item. That is the base quantity intervening in the inequalities introduced further in the method description.

Each nutrient nⱼ may be constrained by two values L₁ and Uⱼ that are respectively the lower and upper limits. If such limits exist, to keep a nutrient nⱼ under target the total nutrient quantity must satisfy the two inequalities:

Tⱼ>L₁ (1a)

Tⱼ<Uⱼ (1b)

Because every nutrient is constrained that way, the system must be formed with at most 2p such inequalities, p being the number of considered nutrients. Together they constitute the Nutrient Target Constraints of the HSP system. The objective of the HSP algorithm is to find a solution that, if resolvable, makes every nutrient nⱼ under target by satisfying all the inequalities (1a) and (1b).

Besides, the Swap Matrix is introduced as a table hosting the items that are candidates to replace S₀. Let's assume that a set of k food candidates {S₁, S₂, . . . , Sₖ} exists. The nutrient composition is known for every food candidate S₁. These quantities denoted Nᵢⱼ are set in the following table:

TABLE 6
tabular representation of the initial state of the HSP problem

| | n₁ | n₂ | . . . | nₚ |
|---|---|---|---|---|
| S₁ | N₁₁ | N₁₂ | . . . | N₁ₚ |
| S₂ | N₂₁ | N₂₂ | . . . | N₂ₚ |
| S₃ | N₃₁ | N₃₂ | . . . | N₃ₚ |
| . . . | . . . | . . . | . . . | . . . |
| Sₖ | Nₖ₁ | Nₖ₂ | Nₖⱼ | Nₖₚ |

For instance, the food S₁ is composed of N₁₁ units of nutrient n₁ plus N₁₂ units of n₂, etc. The highlighted cells define the k×p Swap Matrix.

With the input parameters stated, we can now introduce and relate the variables of the HSP system. The idea is to create a map associating each candidate food Sᵢ to a Boolean variable xᵢ. If xᵢ=1 then the substitute Sᵢ is a solution to replace the initial food S₀. Contrariwise, xᵢ=0 means that Sᵢ is not a solution.

Because the HSP system is constrained to choose one, and only one, substitute Sᵢ, only one single variable xᵢ is allowed to have a value=1. That constraint can be expressed mathematically with the following equality:

$$\begin{matrix}{x}_{1} + {x}_{2} + … + {x}_{k} = 1 & where {x}_{i} ∈ \{ 0 , 1\end{matrix} \} \qquad ( 2 )$$

That equality is the fundamental equation of the HSP system called swap equation. If it does not have any solution then it is not possible to substitute S₀. If at least one solution xᵢ=1 exists then the food Sᵢ can replace the initial food S₀.

Let's now revisit the inequalities (1a) and (1b) in order to plug the variables xⱼ into it. If Sᵢ substitutes S₀ then the total nutrient quantity Tⱼ is changed using the formula below:

Tⱼ=T₀ⱼ−qⱼ+Nᵢⱼ·xᵢ=Qⱼ+Nᵢⱼ·xᵢ

where T₀ⱼ is the initial total quantity before substitution and Tⱼ the total quantity after substitution.

Plugging Tⱼ into (1a) and (1b) gives:

$$\begin{matrix}{Q}_{j} + {N}_{i j} \cdot {x}_{i} > {L}_{j} & ( 3 a ) \\ {Q}_{j} + {N}_{i j} \cdot {x}_{i} < {U}_{j} & ( 3 b )\end{matrix}$$

where i∈{1, 2, . . . , k} identifies a food candidate whereas j∈{1, 2, . . . , p} identifies a nutrient. Because every candidate food for each nutrient must satisfy (3a) and (3b) in addition to the swap equality, the HSP system forms a k-variable system of 2.k.p inequalities such as below:

$$\{ \begin{matrix}{x}_{1} + {x}_{2} + … + {x}_{k} = 1 \\ {Q}_{1} + {N}_{11} \cdot {x}_{1} > {L}_{1} \\ {Q}_{1} + {N}_{11} \cdot {x}_{1} < {U}_{1} \\ {Q}_{1} + {N}_{21} \cdot {x}_{2} > {L}_{1} \\ {Q}_{1} + {N}_{21} \cdot {x}_{2} < {U}_{1} \\ ⋮ \\ {Q}_{1} + {N}_{k 2} \cdot {x}_{k} > {L}_{2} \\ {Q}_{1} + {N}_{k 2} \cdot {x}_{k} < {U}_{2} \\ {Q}_{2} + {N}_{12} \cdot {x}_{1} > {L}_{2} \\ {Q}_{2} + {N}_{12} \cdot {x}_{1} < {U}_{2} \\ ⋮ \\ {Q}_{j} + {N}_{i j} \cdot {x}_{i} > {L}_{j} \\ {Q}_{j} + {N}_{i j} \cdot {x}_{i} < {U}_{j} \\ ⋮ \\ {Q}_{p} + {N}_{k - 1 , p} \cdot {x}_{k - 1} > {L}_{p} \\ {Q}_{p} + {N}_{k - 1 , p} \cdot {x}_{k - 1} < {U}_{p} \\ {Q}_{p} + {N}_{k p} \cdot {x}_{k} > {L}_{p} \\ {Q}_{p} + {N}_{k p} \cdot {x}_{k} < {U}_{p}\end{matrix} \qquad ( HSP )$$

The following section shows how to resolve that system using simple examples.

### A. Examples

### 1. Example 1: One Solution

Let's consider a shopping cart of food items composed of 3 nutrients: n₁, n₂ and n₃. The goal is to reduce the quantity of nutrient n₁, constrained by n₂ and n₃ that cannot be degraded because they are more important than n₁. The three nutrients are constrained with an arbitrary upper and lower limit as defined below:

n₁<14

n₁>10

n₂<8

n₃<10

Let's start with defining the initial Nutrient Matrix with arbitrary values as below.

TABLE 7
exemplar initial state

| | n₁ | n₂ | n₃ |
|---|---|---|---|
| T | 16 | 7 | 7 |
| S₀ | 10 | 3 | 0 |
| T − S₀ | 6 | 4 | 7 |

Note that the total nutrient n₁ has a total quantity T=16 that is greater than the upper limit 14. The other nutrients n₂ and n₃ (in green) are under target. So, the goal of that example is to substitute S₀ with a food that will make n₁ less than 14 while keeping n₂ and n₃ under their limit, respectively 8 and 10.

Let's assume in the example that 5 possible food substitutes have been identified with a composition as shown in the Swap Matrix below.

TABLE 8
exemplar Swap Matrix

| | n₁ | n₂ | n₃ |
|---|---|---|---|
| S₁ | 8 | 0 | 2 |
| S₂ | 4 | 6 | 1 |
| S₃ | 6 | 1 | 2 |
| S₄ | 2 | 3 | 8 |
| S₅ | 7 | 3 | 5 |

In that example, S₁ is composed of 8 units of nutrient n₁, has no n₂ and has 2 units of n₃. Similarly, S₂ is composed of 4 units of n₁, 6 units of n₂ and 1 unit of n₃, and so on.

Let's apply the advanced HSP algorithm. For doing that we need first to form the HSP system. The resolution is expected to be determined by computing the value of the 5 Boolean variables x₁, x₂ . . . x₅ that must satisfy the equation (2):

$$\begin{matrix}{x}_{1} + {x}_{2} + {x}_{3} + {x}_{4} + {x}_{5} = 1 & where {x}_{i} ∈ \{ 0 , 1 \}\end{matrix} \qquad ( 2 )$$

The second step is to build the inequalities (3) for the 3 nutrients contained in the 5 possible food substitutes:

Qⱼ+Nᵢⱼ·xᵢ<Uⱼ where j∈{1,2,3} and i∈{1,2,3,4,5}

Qⱼ+Nᵢⱼ·xᵢ>Lⱼ where j∈{1,2,3} and i∈{1,2,3,4,5}

Let's start with specifying that the nutrient n₁ in food S₁ must be less than U₁=14. The Nutrient Matrix provides Q₁=6 and the Swap Matrix gives N₁₁=8, hence:

6+8x₁<14 (1.1a)

6+8x₁>10 (1.1b)

The same process is used for building the other inequalities. It comes:

6+4x₂<14 (1.2a)

6+4x₂>10 (1.2b)

6+6x₃<14 (1.3a)

6+6x₃>10 (1.3b)

6+2x₄<14 (1.4a)

6+2x₄>10 (1.4b)

6+7x₅<14 (1.5a)

6+7x₅>10 (1.5b)

There are no lower limit constraints on the nutrients 2 and 3 then just “less” inequalities of type (3a) are required:

4+0x₁<8 (1.6)

4+6x₂<8 (1.7)

4+x₃<8 (1.8)

4+3x₄<8 (1.9)

4+3x₅<8 (1.10)

7+2x₁<10 (1.11)

7+x₂<10 (1.12)

7+2x₃<10 (1.13)

7+8x₄<10 (1.14)

7+5x₅<10 (1.15)

Note importantly that several of these inequalities are trivial because they are always satisfied regardless of the x variable value. For instance, the inequality (1.2): 6+4x₂<14 is trivial; it therefore can be removed from the system because plugging x₂=0 (yielding 6<14) or x₂=1 (yielding 10<14) in it makes no difference: the inequality holds true in both cases.

The removal of every such inequality is significantly simplifying the overall system, which is left with only 7 inequalities below:

6+8x₁<14 (1.1a)

6+8x₁>10 (1.1b)

6+6x₃>10 (1.3b)

6+7x₅>10 (1.5b)

4+6x₂<8 (1.7)

7+8x₄<10 (1.14)

7+5x₅<10 (1.15)

Note that the two inequalities (1.1a) and (1.1b) are mutually incompatible; if the first is satisfied (xᵢ=0) then the second can't be and vice versa. Such mutual incompatibility exists between (1.5b) and (1.15). x1 and x5 are undetermined, so S₁ and S₅ are excluded solutions. The overall system can be further reduced by removing the incompatible inequalities.

6+6x₃>10 (1.3b)

4+6x₂<8 (1.7)

7+8x₄<10 (1.14)

That reduced HSP system becomes straight to resolve. Clearly the only way to hold (1.3b) true is when setting x₃=1. Likewise, (1.7) is held true only if x₂=0 and (1.14) are held true only if x₄=0. The equation (2) is then satisfied with the only solution:

x₃=1

The HSP system has therefore one single solution: x₃=1. That is interpreted as “S₃ can substitute S₀”, moving n₁ between lower and upper limit and, at the same time, keeping n₂ and n₃ under target.

### 2. Example 2: Non-Resolvable HSP System

The previous example was nicely picked. One single solution exists. Let's now modify slightly the swap matrix as below to observe the dramatic consequences on the system resolution.

TABLE 9
another exemplar Swap Matrix

| | n₁ | n₂ | n₃ |
|---|---|---|---|
| S₁ | 8 | 0 | 2 |
| S₂ | 4 | 6 | 1 |
| S₃ | 9 | 1 | 2 |
| S₄ | 2 | 3 | 8 |
| S₅ | 7 | 3 | 5 |

The n₁ quantity in S₃ is now 9 instead of 6. That simple change alone results in the construction of a non-resolvable HSP system. Indeed, the equation (1.3), previously trivial, has changed as below and can no longer be ignored in the reduced system:

6+9x₃<14 (1.3′)

The reduced system becomes non-resolvable because that is forcing x₃=0, additional condition making impossible to meet the swap equation (2).

x₁+x₂+x₃+x₄+x₅=1 (2)

6+8x₁<14 (1.1)

6+9x₃<14 (1.3′)

4+6x₂<8 (1.7)

7+8x₄<10 (1.14)

7+5x₅<10 (1.15)

### 3. Example 3: Multiple Solutions

The previous example 2 was a slightly change of the initial example 1. Let's reuse the example 1 again but after reducing the n₁ quantity in S₁ from 8 to 7 units. The swap matrix is changed as below:

TABLE 10
another exemplar Swap Matrix

| | n₁ | n₂ | n₃ |
|---|---|---|---|
| S₁ | 7 | 0 | 2 |
| S₂ | 4 | 6 | 1 |
| S₃ | 6 | 1 | 2 |
| S₄ | 2 | 3 | 8 |
| S₅ | 7 | 3 | 5 |

This new matrix has an impact on the initial inequality (1.1) that is changed to 6+7x₁<14 (1.1′) and has now become trivial; therefore the reduced system can ignore (1.1) and be simplified as shown below:

$$\begin{matrix}{x}_{1} + {x}_{2} + {x}_{3} + {x}_{4} + {x}_{5} = 1 & ( 2 ) \\ 4 + 6 {x}_{2} < 8 & ( 1.7 ) \\ 7 + 8 {x}_{4} < 10 & ( 1.14 ) \\ 7 + 5 {x}_{5} < 10 & ( 1.15 )\end{matrix}$$

x₂, x₄ and x₅ are constrained to 0, leaving the swap equation as:

x₁+x₃=1 (2′)

Equation (2′) has clearly two solutions: (x₁, x₃)∈{(0,1), (1,0)}, which can be interpreted as S₁ or S₃ can be a substitute of S₀.

### B. How to Mitigate a Non-Resolvable HSP?

The example 2 shows a non-resolvable HSP system. However, that is possible to change a condition in some inequalities to augment the likelihood of finding a solution. This happens often in practice. As the example 2 has shown, that is not always possible to find a food substitute that makes a nutrient under target. More modestly, if that case occurs, one should seek a substitute that enhances the nutrient by moving its quantity closer to the target without necessarily forcing it to be lower than the official (dietary) upper limit.

So, let's just do that with the example 2 by changing the limit of nutrient n₁, just enough to enhance it, by setting the threshold as the initial quantity instead of the upper limit. There are k inequalities impacted by that change but let's focus on (1.3), that is:

6+9x₃<16 (1.3″) instead of:

6+9x₃<14 (1.3′)

Unlike the former inequality (1.3′), (1.3″) is trivial, always satisfied regardless of x₃ value (x₃=1 still makes it <16). The removal of (1.3″) makes the reduced system solvable, actually the same as example 1 whose outcome is the solution: x₃=1.

Conclusion, the substitution of S₀ with S₃ does not succeed in making n₁ under target, still which is enhancing the basket of food F by moving n₁ closer to its target without impacting negatively n₂ and n₃.

The method 1900 of the basic Healthy Swap HS method is depicted in FIG. 19. The method 1900 seeks alternative food items in order to substitute (swap) an original item (F) that contributes negatively to a given nutrient (N). That method can be assimilated to the resolution of the HSP system such as described in detail in the previous pages. It is to be understood that the HSP problem does not always have solutions. If there is none, then the original item is not swappable; a possible outcome of the HS method 1900 is the impossibility to improve a given nutrient by substituting a targeted food item.

The high-level steps of the method 1900 are described next. The initial step 1910 consists of receiving the four input arguments required for the method:

1) Health Cart (HC) 400 as a possible representation is shown in FIG. 6

2) Shopping Cart (SC) 510a as a possible representation is shown in FIG. 7A

3) The item (F) targeted to be swapped

4) The deficient nutrient (N) targeted to be improved

The next step 1920 identifies the nutrients to include into the HSP system. Those are generally the nutrients having more importance than the targeted nutrient N. It is to be understood that any replacement of the item F by another shall not lower the score of any nutrient included in the system. The next step 1930 creates the nutrient matrix [NM] such as described in the table 5. The Tⱼ values are provided by the Health Cart section 402a (key AverageDailyAmount) calculated by the step 570 in the method 500 shown in FIG. 7, for every nutrient selected in step 1920.

The qⱼ values are calculated similarly as processed by the step 520 in the method 500 for computing the HC and described using an example through the lines 10-15 at page 27. The Uⱼ and Lⱼ limit values are provided by the Health Cart section 402a keys: UpperLimit and LowerLimit. The terms Qⱼ=Tⱼ−qⱼ, as well as the Uⱼ and Lⱼ values collected above are memorized in a specific container keyed by Nutrient Id. The next step 1940 seeks potential food candidates to replace the item F. Each food candidates in {S₁, S₂, . . . , Sₖ} must be of the same food type as the original item F. In practice, the objective is to, for example, replace a particular high-fat cheese item with possible low-fat cheeses by making sure that it is not replaced with a low-fat yogurt. That is the reason why the method is founded on a specific food taxonomy configured in the system, such as the exemplar one shown in FIG. 20A. For each food in the set {S₁, S₂, . . . , Sₖ} the process 1940 creates an object which computes internally the Nᵢⱼ quantities, which are memorized in a container keyed by a pair (Food Id, Nutrient Id). Further, the process 1940 is to restrict that initial set {S₁, S₂, . . . , Sₖ} to a subset by keeping only the candidates that are effectively improving the nutrient. There is a HS selection process in charge of keeping the items among that subset that improve the nutrient deficiency. If the original item contains too much of the nutrient then only the items composed of less are kept. Contrariwise, if the original item does not contain enough of the nutrient then the items composed of more are kept.

Furthermore, the HS selection process is taking into account the impact of the substitute over the nutrient deficiency. In order to know whether an item has more or less nutrient amount than the original one, the HS selection process relies on the existing food composition master 2231 in FIG. 4C providing the nutrient amount of every food item.

Then, for each selected food candidate Sᵢ, (YES to step 1945), the step 1950 generates the swap matric [SW] such as the exemplar illustrated in the Table 6. Then the step 1960 builds the inequalities (3a) and (3b). There are as many inequalities as elements in the Swap Matrix created in the previous step. The trivial inequalities defined by the conditions Qⱼ+Nᵢⱼ<Uⱼ or Nᵢⱼ>Lⱼ are flagged in the container created in step 1950.

Qⱼ, Uⱼ and Lⱼ are looked up with a Nutrient Id. Nᵢⱼ are looked up with the pair (Food Id, Nutrient Id). The step 1970 seeks the solution xᵢ satisfying the swap equations (2) and all the non-trivial inequalities (3a) and (3b) not flagged in the previous step 1960. If there is at least one solution (YES to step 1975) then a CPR record is created for every solution in step 1980. All the CPR records are piled up into an array of CPR's 1990. Each record is mapped to a food item Sᵢ which can be selected by a shopper to replace the item F in the shopping cart. If there are no food candidates (NO to step 1945) or no solution is found (NO to step 1975), then the method ends with no solution to swap the item.

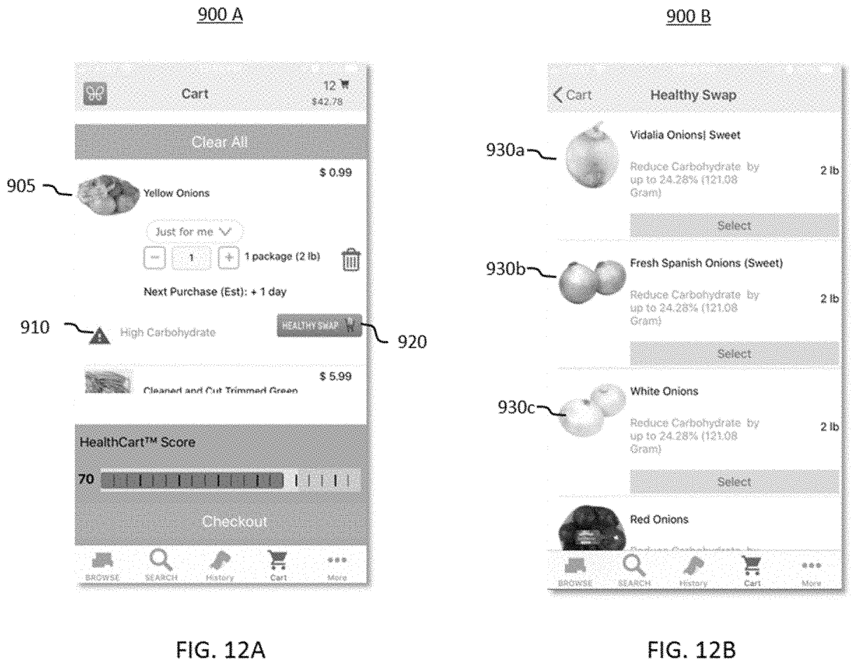

The method 1900 shown in the flowchart of FIG. 19 is triggered by the means of a selection of, for example, the button 920 shown in the user interface 900A in FIG. 12A. The output is a list of substitute food items to the original item F 905, each substitute is improving the given nutrient N nudged with the alert icon 910, possibly moving it within target, guaranteeing that no other more important nutrients are degraded. At the end of the method 1900, the system will leave the end-user to select one of the possible substitutes 930a, 930b, and 930c such as displayed like an exemplar user interface 900B shown in FIG. 12B. For the sake of convenience, the HCMS system 200 allows the list of substitutes to be sorted by different means, like by nutritional impact—making the nutrient within, or the closest to, target—by price or by other criteria such as personal food preference among others. It is to be noticed that combining different sorting is configurable in the HCMS system 200, including external criteria such as defined by grocers aiming at incenting shoppers to purchase certain targeted food items.

The generalized HS method 1700 in FIG. 17 is aimed at replacing all the shopping cart items contributing negatively to a given nutrient. An example of utilization would be when there is a HC showing too much carbohydrates due to many food items in a shopping cart; there is a convenience here to apply the generalized HS method that would suggest a list of substitutes to all these items. FIG. 17 depicts the flowchart of a generalized HS method 1700. The first step 1710 receives the three input arguments of the method:

1) Health Cart (HC) 400 as a possible representation is shown in FIG. 6

2) Shopping Cart (SC) 510a as a possible representation is shown in FIG. 7A

3) The deficient nutrient (N) targeted to be improved

The next step 1720 is to identify all the food items in the shopping cart that can be substituted given the nutrient (N) to improve. That process is described as the nudging process, discussed previously. For each shopping cart item F identified in the previous step (YES to step 1725), the step 1730 is to consider the next item F and trigger the basic HS method 1900 given the item F as argument, in addition to the HC, SC and nutrient N. When all the items F have been exhausted (No to step 1725), the method 1700 generates a list of substitutes 1750 for each food items identified in step 1720. As for the basic HS, the system leaves the end-user to pick the substitutes for each food item or to leave the system to pick them on his behalf based on some criteria such as impact, price among others.

The overarching and ultimate HS method 1800 is aimed at replacing every shopping cart item contributing to any of the deficient nutrients in a HC. The method 1800 optimizes the healthfulness of an entire shopping cart by the means of raising the overall HC score as high as possible. A HC is optimum when every nutrient is within target, or if not possible, as close as possible to its upper or lower limit. The overall flowchart of the ultimate HS method 1800 is shown in FIG. 18. The first step 1810 receives the two input arguments required for the method:

1) Health Cart (HC) 400 as a possible representation is shown in FIG. 6

2) Shopping Cart (SC) 510a as a possible representation is shown in FIG. 7A

The next step 1820 identifies all the deficient nutrients, the ones that are out of targets and, therefore, contribute the most to lower the overall HC score. The overall method 1700 described in the previous section is next repeated for each deficient nutrient (YES to step 1825), the most important nutrient first and then the next most important and so on until the deficient nutrients are all processed (NO to step 1825). The outcome of the method 1800 is a healthier shopping cart 1830 filled in with similar food items than the initial one, some of the latter replaced so that the most important nutrients are all in target or closer to it.

The basic HS method 1900, in particular the step 1940, is based on the classification of foods into categories of the same kind. As an example, a simple excerpt of food taxonomy 2010 is shown in FIG. 20A in which food is broken down into three exemplar categories: Fruit 2011, Meat 2012 and Dairy 2013. Each of those can themselves be broken down further, for example Fruit 2011 into Apple 20111, Orange 20112 and Banana 20113, among several other sorts of fruits. Many food taxonomy schemes can exist for fulfilling different purposes. The HCMS system 200 is designed to handle many food taxonomies. Some of them may be dedicated to the HS method 1900. The sub data model 224 in FIG. 4D shows how the HCMS system 200 can store several food taxonomies. The system table: Food Group (2242) contains every node constituting a food taxonomy: Beef, Apple, Cheese, Meat, etc. are examples of four food groups loaded as shown in the following table 10.

TABLE 10
table Food Group

| Food | Food | Food | Food | Parent | Parent Food |
|---|---|---|---|---|---|
| Group | Group | Group | Textual | Food | Group |
| Id | Type Id | Description | Fact | Group Id | Type Id |
| 1 | 1 (HS Taxo) | Meat | Quis Est | | 1 (HS Taxo) |
| 2 | 1 (HS Taxo) | Beef | Quis Est | 1 | 1 (HS Taxo) |
| 3 | 1 (HS Taxo) | Apple | Quis Est | 21 | 1 (HS Taxo) |
| 4 | 1 (HS Taxo) | Cheese | Quis Est | 22 | 1 (HS Taxo) |

Note the self-reference relationship 22421 of the table 2242 in FIG. 4D; that relationship allows food groups to form a hierarchy. For instance, Beef identified with the Food Group id=2 in the second row of the table 10 is associated to the Parent Food Group Id=1, which is Meat according to the first row of the table 10. The top two rows are linked together through the Parent Food Group Id, forming thereby the parent-child relationship: Meat-Beef, materializing thereby that beef is a sort of meat. Each food croup can belong to one or many food group types that are defined in the system table 2243. That table stores the different food taxonomy schemes configured in the system, for instance Healthy Swap Food Taxonomy 2010 in FIG. 20A, Diet Food Taxonomy 2020 in FIG. 20B, Food Nutrition Taxonomy, etc. The system table: Food Category (2241) allows the system to allocate food items to food groups, generally the to the leaf nodes of the hierarchy. A food group can contain many food items and a given food item can be part of several food groups (nodes).

A specific method exists in the HC computing process 500 in case of a lack of nutrient. Vitamins or minerals, for instance, are never present in some kind of food. The lack of it should not be a reason to nudge a food item systematically. In that case, the way to higher the nutrient amount is sometimes to simply add an item to the shopping cart. For example, it would not be appropriate to nudge chicken like “low in fiber” because meat in general is not composed of fiber. The addition of fruits such as pears or apples to the shopping cart, for instance, is a better way to higher the fiber amount rather than attempting to seek a meat alternative to chicken. The next section describes a method to seek supplemental items to add to the shopping cart with the goal of increasing the quantity of a particular high-is-better or neutral nutrient.

There is a method to recommend additional food to a shopping cart in order to increase the content of some deficient nutrient, generally of class: high-is-better, but not limited to. That method relies on a specific food taxonomy that is configured in the system. An exemplar representation of such a food taxonomy is depicted in FIG. 20B. The food taxonomy 2020 is nutrition driven. The food categories in it are classified according to the richness of nutrient. FIG. 20B shows an excerpt of an exemplar nutritional food taxonomy 2020. The highest nodes under the root represent nutrients such as Carbohydrate 2110, Calcium 2111, Fiber 2112, etc. The descendent nodes are food categories that are rich in the parent nutrient. For instance, bread 2120 and starchy vegetables 2121 are both rich in carbohydrate and therefore under the node 2110. The HCMS system 200 leverages that food taxonomy to suggest food items that can be added to a shopping cart in order to increase a given nutrient content. For instance, the system can provide the advice regarding a low-in-fiber shopping cart that could be advantageously supplemented with fruits such as bananas or oranges. After the deficient nutrient is identified the system looks up the food taxonomy such as 2020 to seek the deficient nutrient node (e.g. fiber 2112) in it. All the food categories and/or food items underneath such as banana or bread can be exposed to the end-users as possible supplemental foods. If such items are already in the shopping cart, then the system can recommend buying more of them. Ultimately, the daily quantity of specific food item can be computed given the gap between he LL and the current daily fiber amount in the HC.

While some of the embodiments disclosed above are described with examples of a shopping cart or a grocery cart, the same or similar embodiments may be used for evaluating a meal or a collection of meals using the systems and methods of the present invention.

Subsets or combinations of various embodiments described above provide further embodiments.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

What is claimed is:

1. A method executed by a programmed data processing device system of a cart management system, the method comprising:
receiving item information of one or more items in the cart, the item information including a quantity of each item of the one or more items in the cart and a sharing parameter for at least one item of one or more items in the cart;
receiving profile information of a consumer of the one or more items in the cart, the profile information including diagnostic information of at least one medical condition of the consumer;
receiving dietary reference intake information of one or more nutrients, the dietary reference intake information including a recommended intake value for each nutrient of one or more nutrients;
computing a daily consumption value for each item of the one or more items in the cart;
generating, for each nutrient of the one or more nutrients, a nutrient score by:
adjusting the recommended intake value of the nutrient based at least on the diagnostic information of the at least one medical condition of the consumer;
computing a daily nutrient consumption value for the nutrient based on all items in the cart and the respective daily consumption quantity values for all items in the cart; and
generating the nutrient score based at least on the daily nutrient consumption value for the nutrient and the adjusted recommended intake value of the nutrient; and
generating the health cart score by computing a weighted mean of the nutrient scores for each nutrient of the one or more nutrients.

2. The method according to claim 1, further comprising providing an indication of the generated health cart score to the consumer.

3. The method according to claim 1, further comprising outputting a health cart document including at least the generated health cart score, the nutrient scores for each nutrient of the one or more nutrients, and the adjusted recommended intake value for each nutrient of the one or more nutrients.

4. The method according to claim 1, wherein the sharing parameter includes at least one of a family sharing factor or a sharing option.

5. The method according to claim 1, wherein the receiving dietary reference intake information of one or more nutrients includes adjusting the dietary reference intake information based at least on one factor of one or more factors included in the profile information of the consumer of the one or more items in the cart, the one or more factors including an age of the consumer, a weight or the consumer, a height of the consumer, an activity level of the consumer, or a basal metabolic rate (BMR) of the consumer.

6. The method according to claim 1, wherein the adjusting the recommended intake value of the nutrient includes computing an eta-coefficient to increase or decrease the recommended intake value of the nutrient by a predetermined factor based on the diagnostic information of the at least one medical condition of the consumer.

7. The method according to claim 1, wherein the computing the daily consumption value for each item of the one or more items in the cart includes, for each item:
in a case where the item information does not include a sharing parameter for an item of the one or more items in the cart, setting a daily consumption quantity value of the item to the quantity of the item; and
in a case where the item information includes a sharing parameter for an item of the one or more items in the cart, computing the daily consumption quantity value for the item based at least on the quantity of the item and the sharing parameter for the item.

8. The method according to claim 1, further including:
identifying at least one deficient nutrient based on the nutrient scores of the one or more nutrients;
identifying at least one item of the one or more items in the cart contributing to the nutrient score of the deficient nutrient;
replacing the identified item in the cart with another item to improve the nutrient score of the deficient nutrient; and
recalculating the health cart score.

9. The method according to claim 1, further including:
computing an item retention for each item of the one or more items in the cart, each item retention defining a consumption period for a respective item of the one or more items in the cart; and
adjusting the daily consumption quantity value of each item of the one or more items in the cart based at least on the respective item retention.

10. The method according to claim 9, wherein computing the item retention for each item of the one or more items in the cart includes:
in a case where a purchase history of the item is available, computing the item retention based at least on the purchase history of the item; and
in a case where the purchase history of the item is unavailable, computing the item retention based at least on a serving size of the item.

11. A health cart management system comprising:
an input-output device system communicatively connected to a display device system;
a memory device system storing a program; and
a data processing device system communicatively connected to the input-output device system and the memory device system, the data processing device system configured at least by the program at least to:
receive item information of one or more items in the cart, the item information including a quantity of each item of the one or more items in the cart and a sharing parameter for at least one item of one or more items in the cart;
receive profile information of a consumer of the one or more items in the cart, the profile information including diagnostic information of at least one medical condition of the consumer;
receive dietary reference intake information of one or more nutrients, the dietary reference intake information including a recommended intake value for each nutrient of one or more nutrients;
compute a daily consumption value for each item of the one or more items in the cart;
generate, for each nutrient of the one or more nutrients, a nutrient score by:
adjusting the recommended intake value of the nutrient based at least on the diagnostic information of the at least one medical condition of the consumer;
computing a daily nutrient consumption value for the nutrient based on all items in the cart and the respective daily consumption quantity values for all items in the cart; and
generating the nutrient score based at least on the daily nutrient consumption value for the nutrient and the adjusted recommended intake value of the nutrient; and
generate the health cart score by computing a weighted mean of the nutrient scores for each nutrient of the one or more nutrients.

12. The health cart management system according to claim 11, wherein the data processing device system is configured at least by the program at least to:
provide an indication of the generated health cart score to the consumer.

13. The health cart management system according to claim 11, wherein the data processing device system is configured at least by the program at least to:
output a health cart document including at least the generated health cart score, the nutrient scores for each nutrient of the one or more nutrients, and the adjusted recommended intake value for each nutrient of the one or more nutrients.

14. The health cart management system according to claim 11, wherein the sharing parameter includes at least one of a family sharing factor or a sharing option.

15. The health cart management system according to claim 11, wherein the data processing device system is configured at least by the program at least to:
adjust the received dietary reference intake information based at least on one factor of one or more factors included in the profile information of the consumer of the one or more items in the cart, the one or more factors including an age of the consumer, a weight or the consumer, a height of the consumer, an activity level of the consumer, or a basal metabolic rate (BMR) of the consumer.

16. The health cart management system according to claim 11, wherein the data processing device system is configured at least by the program at least to:
adjust the recommended intake value of the nutrient by computing an eta-coefficient to increase or decrease the recommended intake value of the nutrient by a predetermined factor based on the diagnostic information of the at least one medical condition of the consumer.

17. The health cart management system according to claim 11, wherein the data processing device system is configured at least by the program at least to:
for each item, compute the daily consumption value for each item of the one or more items in the cart by:
in a case where the item information does not include a sharing parameter for an item of the one or more items in the cart, setting a daily consumption quantity value of the item to the quantity of the item; and
in a case where the item information includes a sharing parameter for an item of the one or more items in the cart, computing the daily consumption quantity value for the item based at least on the quantity of the item and the sharing parameter for the item.

18. The health cart management system according to claim 11, wherein the data processing device system is configured at least by the program at least to:
identify at least one deficient nutrient based on the nutrient scores of the one or more nutrients;
identify at least one item of the one or more items in the cart contributing to the nutrient score of the deficient nutrient;
replace the identified item in the cart with another item to improve the nutrient score of the deficient nutrient; and
recalculate the health cart score.

19. The health cart management system according to claim 11, wherein the data processing device system is configured at least by the program at least to:
compute an item retention for each item of the one or more items in the cart, each item retention defining a consumption period for a respective item of the one or more items in the cart; and
adjusting the daily consumption quantity value of each item of the one or more items in the cart based at least on the respective item retention.

20. The health cart management system according to claim 19, wherein the data processing device system is configured at least by the program at least to:
compute the item retention for each item of the one or more items in the cart by:
in a case where a purchase history of the item is available, computing the item retention based at least on the purchase history of the item; and
in a case where the purchase history of the item is unavailable, computing the item retention based at least on a serving size of the item.
